(19) United States
(12) Patent Application Publication
(10) Pub. No.: US 2016/0178562 A1
(43) Pub. Date: Jun. 23, 2016

(54) COMPETITIVE ENZYMATIC ASSAY

(71) Applicant: OHMX Corporation, Evanston, IL (US)

(72) Inventor: Adam G. Gaustad, Kansas City, MO (US)

(73) Assignee: OHMX Corporation, Evanston, IL (US)

(21) Appl. No.: 14/975,669

(22) Filed: Dec. 18, 2015

Related U.S. Application Data
(60) Provisional application No. 62/094,934, filed on Dec. 19, 2014.

(51) Int. Cl. G01N 27/327; C12Q 1/00

(52) U.S. Cl. CPC G01N 27/3275; C12Q 1/001

(57) ABSTRACT

Competitive assays are provided for the detection and quantification of a target analyte utilizing a modified electro-active moiety and an enzyme, in which the target analyte and a target analog moiety are substrates. This method may be used to detect and/or quantify many classes of biological molecules and has a number of applications, e.g., in vitro diagnostic assays and devices.

### RELATED APPLICATIONS

This application claims the benefit under 35 U.S.C. §119 of U.S. provisional application 62/094,934, filed Dec. 19, 2014, the entire contents of which are incorporated herein by reference.

### FIELD OF THE INVENTION

Competitive assays are provided for the detection and quantification of a target analyte utilizing a modified electro-active moiety and an enzyme, in which the target analyte and a target analog moiety are substrates. This method may be used to detect and/or quantify many classes of biological molecules and has a number of applications, e.g., in vitro diagnostic assays and devices.

### BACKGROUND OF THE INVENTION

The electromotive force (EMF) is the maximum potential difference between two electrodes of a galvanic or voltaic cell, where the standard hydrogen electrode is on the left-hand side for the following cell:

| | | | | |
|---|---|---|---|---|
| 1 | | | | 2 |
| Pt Electrode | H₂ | Aqueous Electrolyte | 10⁻³ M Fe(ClO₄)₃ | Pt |
| | | Solution | 10⁻³ M Fe(ClO₄)₂ | |

The EMF is called the electrode potential of the electrode placed on the right-hand side in the graphical scheme of the cell, but only when the liquid junction between the solutions can be neglected or calculated, or if it does not exist at all.

The electrode potential of the electrode on the right-hand side (often called the oxidation-reduction potential) is given by the Nernst equation:

E_{Fe}_{³⁺}_{/Fe}_{²⁺}=E_{Fe}_{³⁺}_{/Fe}_{²⁺}⁰+(RT/F)ln(a_{Fe}_{³⁺}/a_{Fe}_{²⁺}) (Eq. 1)

where R is the universal gas constant (8.31447 Jmol⁻¹K⁻¹), T is the temperature in Kelvin, F is the Faraday constant (9.64853×10⁴ Coulombs).

This relationship follows from equation 2 (Eq. 2) when (μ_{Fe}_{^{3−}}⁰−μ_{Fe}_{²⁺}⁰)/F is set equal to E_{Fe}_{³⁺}_{/Fe}_{²⁺}⁰ which is the standard electrode potential, and the pH and ln p_{H}_{₂} are equal to zero.

E_{Fe}_{³⁺}_{/Fe}_{²⁺}=(μ_{Fe}_{^{3−}}⁰−μ_{Fe}_{²⁺}⁰)/F+(RT/F)pH+(RT/F)ln(p(H₂)a_{Fe}_{³⁺}/p⁰a_{Fe}_{²⁺}) (Eq. 2)

In the subscript of the symbol for the electrode potential, E, the symbols for the oxidized and reduced components of the oxidation-reduction system are indicated. With more complex reactions it is recommended to write the whole reaction that takes place in the right-hand half of the cell after symbol E (the ‘half-cell’ reaction); thus, in the present case

E_{Fe}_{³⁺}_{/Fe}_{²⁺}≡E(Fe³⁺+e=Fe²⁺).

Quantity E_{Fe}_{³⁺}_{/Fe}_{²⁺}⁰ is termed the standard electrode potential. It characterizes the oxidizing or reducing ability of the component of oxidation-reduction systems. With more positive standard electrode potentials, the oxidized form of the system is a stronger oxidant and the reduced form is a weaker reductant. Similarly, with a more negative standard potential, the reduced component of the oxidation-reduction system is a stronger reductant and the oxidized form a weaker oxidant.

The standard electrode E0, in its standard usage in the Nernst equation, equation is described as:

$$E = {E}^{0} + \frac{2.3 RT}{nF} log \frac{{C}_{0} ( 0 , t )}{{C}_{R} ( 0 , t )} \qquad Eq . 3$$

where E⁰ is the standard potential for the redox reaction, R is the universal gas constant (8.314 JK⁻¹mol⁻¹), T is the Kelvin temperature, n is the number of electrons transferred in the reaction, and F is the Faraday constant (9.64853×104 coulombs). On the negative side of E⁰, the oxidized form thus tends to be reduced, and the forward reaction (i.e., reduction) is more favorable. The current resulting from a change in oxidation state of the electroactive species is termed the faradaic current.

### SUMMARY OF THE INVENTION

Specialized electro-active moieties containing functional groups can be designed for use in many detection schemes including self-assembled monolayers, chemical interactions, redox reactions, binding interactions, competitive assays, binding assays, and enzymatic assays. Applications for electro-active moieties have been demonstrated in e.g. U.S. Pat. Nos. 8,802,391 and 8,530,170, and U.S. patent application Ser. No. 13/952,345, producing reproducible, electronic detection e.g., for proteins, enzymes, small molecules and nucleic acids. The electro-active moieties and methods of their use are incorporated herein by reference in their entirety. In some cases, the electro-active moieties have characteristics allowing the coupling of multiple techniques yielding powerful, unique detection methods. The detection and quantification of small molecules using a self-assembling, electro-active moiety in a competitive enzymatic assay scheme is described herein.

In one aspect, the present invention provides compositions and methods for the detection and quantification of target analytes using self-assembling, electro-active moieties used in a competitive enzymatic assay format. For example, in one embodiment, a fixed concentration of an electro-active moiety (EAM) with at least a portion of the structure mimicking that of the target analyte of interest (e.g., target analog moiety or TAM) is introduced into the assay mixture containing a sample. An enzyme that has the target analyte as a substrate may also be introduced into the assay mixture. In such embodiments, the enzyme reacts with both the target analyte and the electro-active moiety comprising the mimic of the target analyte (e.g., target analog moiety or TAM) at a rate dependent upon the concentration of the target analyte in the sample. For example, if more target analyte is present, more target analyte will be reacted and less of the mimic of the target analyte (e.g., target analog moiety or TAM), which is part of the electro-active moiety, will be catalyzed by or otherwise react with the enzyme. Accordingly, if less target analyte is present then the opposite will be true. The amount of reacted and/or unreacted electro-active moiety can be measured and correlated to the amount of target analyte in the sample.

In another aspect, a method for detecting one or more target analytes in a test sample is provided, said method comprising:
(a) contacting the test sample with one or more electroactive moietys (EAMs) and one or more enzymes, wherein each EAM comprises a transition metal complex and an target analog moiety (TAM), each target analyte and target analog moiety being substrates of an enzyme, and each EAM has a first Eo when the TAM has not been modified by the enzyme and a second Eo when at least a portion of the TAM has been modified by the enzyme;
(b) detecting a change between the first Eo and the second Eo of each EAM, wherein the change is an indication of the presence of a target analyte; and
(c) determining the concentration of each target analyte.

In another aspect, a method for detecting one or more target analytes in a test sample is provided, said method comprising:

contacting the test sample with one or more electroactive moietys (EAMs) and one or more enzymes to form an assay mixture in solution phase, wherein each EAM comprises a transition metal complex and an target analog moiety (TAM), each target analyte and target analog moiety are substrates of an enzyme, and each EAM has a first Eo when the TAM has not been modified by the enzyme and a second Eo when at least a portion of the TAM has been modified by the enzyme;
(b) contacting said assay mixture with a solid support comprising an electrode or an array of electrodes under conditions such that one or more self-assembled monolayers (SAMs) forms on said electrode or array of electrodes;
(c) detecting a change between the first Eo and the second Eo of each EAM, wherein said change is an indication of the presence of a target analyte; and
(d) determining the concentration of each target analyte.

In another aspect, a method for detecting one or more target analytes in a test sample is provided, said method comprising:
(a) contacting the test sample with a solid support, said solid support comprising an electrode or an array of electrodes, each electrode comprising:
   (i) a self-assembled monolayer; and
   (ii) one or more covalently attached electroactive moietys (EAMs), each EAM having a first E^{o} and comprising a transition metal complex and a target analog moiety (TAM), wherein the target analyte and the target analog moiety are substrates of an enzyme and wherein each EAM has a second E^{o} when at least a portion of the TAM is modified by the enzyme;
(b) detecting a change between the first Eo and the second Eo of each EAM, wherein said change is an indication of the presence of a target analyte and concentration of the target analyte.

In another aspect, a composition is provided comprising a solid support comprising an electrode or array of electrodes comprising:
(i) one or more self-assembled monolayers (SAMs); and
(ii) one or more covalently attached electroactive active moietys (EAMs) each comprising a transition metal complex and a target analog moiety (TAM), wherein each EAM has a first E0 when said TAM is present and a second E0 when said TAM is modified.

In another aspect, a kit for detecting at least one target analyte in a test sample, comprising any one of the compositions provided herein is provided. In another aspect, a kit for detecting at least two target analytes in a test sample, comprising any one of the compositions provided herein is provided.

In one embodiment of any one of the methods provided herein, an assay mixture in a solution phase is formed in step (a) and prior to step (b).

In one embodiment of any one of the methods provided herein, the method includes contacting an assay mixture with a solid support comprising an electrode or an array of electrodes, under conditions such that a self-assembled monolayer (SAM) forms on said electrode.

In one embodiment of any one of the methods or compositions provided herein, the EAM is covalently attached to the electrode or the array of electrodes on the solid support as the self-assembled monolayer (SAM).

In one embodiment of any one of the methods or compositions provided herein, the EAM further comprises a self-immolative moiety (SIM) which joins said TAM to said transition metal complex.

In one embodiment of any one of the methods or compositions provided herein, the at least one enzyme is selected from the group consisting of proteases, peptidases, phosphatases, oxidases, hydrolases, lyases, transferases, isomerase, ligases, and ligases.

In one embodiment of any one of the methods or compositions provided herein, the transition metal complex comprises a transition metal selected from the group consisting of iron, ruthenium, and osmium.

In one embodiment of any one of the methods or compositions provided herein, the transition metal complex comprises a ferrocene and substituted ferrocene.

In one embodiment of any one of the methods or compositions provided herein, the EAM comprises a flexible oligomer anchor tethering said transition metal complex to said electrode.

In one embodiment of any one of the methods or compositions provided herein, the flexible anchor comprises a hydrophobic oligomer comprising side chains that limit intermolecular hydrophobic interactions and prevent organization and rigidity.

In one embodiment of any one of the methods or compositions provided herein, the EAM comprises a flexible oligomer anchor tethering said transition metal complex to said electrode.

In one embodiment of any one of the methods or compositions provided herein, the flexible anchor comprises an oligomer comprising polar and/or charged functional groups.

In one embodiment of any one of the methods or compositions provided herein, the flexible oligomer anchor tethering said transition metal complex to said electrode comprises poly acrylic acid, polyethylene glycol (PEG), poly vinyl alcohol, polymethacrylate, poly vinylpyrrolidinone, acrylamide, maleic anhydride, poly vinylpyridine, allylamine, ethyleneimine, or oxazoline.

In one embodiment of any one of the methods or compositions provided herein, the electrodes in said array of electrodes are modified with a SAM and wherein at least some of the electrodes comprise a different EAM and TAM from another electrode.

In one embodiment of any one of the methods or compositions provided herein, the different TAMs are substrates for different enzymes.

In one embodiment of any one of the methods provided herein, further comprising detecting two or more different target analytes in said test sample using two or more enzymes.

In one embodiment of any one of the methods or compositions provided herein, the EAM comprises a self-immolative moiety (SIM) that joins the TAM to the transition metal complex.

In one embodiment of any one of the methods or compositions provided herein, the transition metal complex comprises a transition metal selected from the group consisting of iron, ruthenium, and osmium.

In one embodiment of any one of the methods or compositions provided herein, the transition metal complex comprises a ferrocene and substituted ferrocene.

In one embodiment of any one of the methods or compositions provided herein, the EAM comprises a flexible oligomer anchor tethering said transition metal complex to said electrode, said flexible anchor being an oligomer comprising polar or charged functional groups.

In one embodiment of any one of the methods or compositions provided herein, the EAM comprises a flexible oligomer anchor tethering said transition metal complex to said electrode, said flexible anchor being a hydrophobic oligomer comprising side chains that limit intermolecular hydrophobic interactions and prevent organization and rigidity.

In one embodiment of any one of the methods or compositions provided herein, the EAM comprises a flexible oligomer anchor tethering said transition metal complex to said electrode, said flexible anchor being an oligomer comprising poly acrylic acids, polyethylene glycol (PEG), poly vinyl alcohol, polymethacrylate, poly vinylpyrrolidinone, acrylamide, maleic anhydride, and poly vinylpyridine, allylamine, ethyleneimine, or oxazoline.

In one embodiment of any one of the methods or compositions provided herein, solid support further comprises an array of electrodes.

In one embodiment of any one of the methods or compositions provided herein, the electrodes in said array of electrodes are modified with a SAM and wherein at least some of the electrodes comprise a different EAM and TAM from another electrode.

In one embodiment of any one of the methods or compositions provided herein, the different TAMs are substrates of a different enzyme.

In one embodiment of any one of the methods provided herein, the method includes any one of the steps of calculating the ratio of reacted to unreacted EAMs.

In one embodiment of any one of the methods provided herein, the method includes any one of the steps of determining the correlation of the ratio of reacted to unreacted EAMs to the concentration of target analyte.

In one embodiment of any one of the methods provided herein, the method is for determining the concentration of two or more target analytes. In one such embodiment, there are two or more TAMs.

In one embodiment of any one of the methods provided herein, the method comprises or further comprises any one of the steps provided herein.

In one embodiment of any one of the compositions provided herein, the composition comprises or further comprises any one of the features or components provided herein.

In another aspect, any one of the methods or compositions provided herein, including those of the Examples or Figures is provided.

### BRIEF DESCRIPTION OF THE DRAWINGS

FIG. 1 is a schematic of an enzyme utilizing both the target analyte and the electro-active moiety (EAM) comprising the target analog moiety (TAM) as substrates, according to certain embodiments. The higher the target analyte concentration the less target analog moiety will react with the enzyme. Both reacted and unreacted EAM molecules then form a self-assembled monolayer for detection with the amount of reacted and unreacted EAMs correlating to the concentration of target analyte in the sample.

FIG. 2 is a schematic of an exemplary embodiment wherein the concentration of the EAM comprising the target analog moiety is much higher than target analyte concentration. The enzyme reacts with more EAM than target analyte, resulting in a monolayer comprising more reacted EAMs than unreacted EAMs.

FIG. 3 is a schematic of an exemplary embodiment wherein the concentration of the EAM comprising the target analog moiety is much lower than the target analyte concentration. The enzyme reacts with more target analyte than EAM resulting in a monolayer comprising more unreacted EAMs than reacted EAMs.

FIG. 4 is a graph of data gathered for the detection of a target analyte using an EAM comprising the target analog moiety and chymotrypsin as the enzyme. The data fit an exponential curve very well (R=0.9804) with a detection range from low micromolar to low millimolar concentration of target. The target (N-benzoyl-L-tyrosine ethyl ester) was titrated and mixed with EAM comprising the target analog moiety as well as chymotrypsin, allowed to react, and then the reaction mixture was delivered to an electrode for SAM formation prior to detection using cyclic voltammetry. A clear dose response is observed with inverse relationship between target concentration and signal.

FIG. 5 is a graph of a dose response of the target analyte, Tyrosine-ethyl-ester, a substrate for chymotrypsin, in a competitive assay with the EAM (which has a tyrosine attached to the end as a TAM), according to certain embodiments. The samples were run in triplicate and standard deviation error bars are included.

FIG. 6 is a graph of data showing the response of various concentrations of target Lys-Tyr-Lys substrate with 5 uM Chymotrypsin in a competitive assay with the EAM having Tyrosine as the target analog moiety, according to certain embodiments. Line: 0 uM Lys-Tyr-Lys, Square: 50 uM Lys-Tyr-Lys, Asterisk: 200 uM Lys-Tyr-Lys, Circle: 800 uM Lys-Tyr-Lys.

FIG. 7 is a graph of data showing the response of various concentrations of Tyrosine-ethyl-ester substrate with 5 uM Chymotrypsin in competitive assay with the EAM having Tyrosine as the target analog moiety, according to certain embodiments. Line: 125 uM Tyrosine ethyl ester, Square: 500 uM Tyrosine ethyl ester, Asterisk: 1 mM Tyrosine ethyl ester, Circle: 2 mM Tyrosine ethyl ester, Diamond: 4 mM Tyrosine ethyl ester, Triangle: 8 mM Tyrosine ethyl ester with 5 uM Chymotrypsin, 20 min reaction/5 min SAM formation time.

FIG. 8 is a graph of data showing the response of various concentrations of tyrosine ethyl ester substrate with 1.25 uM Chymotrypsin in competitive assay with the EAM having Tyrosine as the target analog moiety, according to certain embodiments. After decreasing the enzyme concentration 4× to 1.25 uM, there was an improvement in the separation of the peaks. Line: 0 uM Tyrosine ethyl ester, Square: 125 uM Tyrosine ethyl ester, Asterisk: 500 uM Tyrosine ethyl ester, Circle: 2 mM Tyrosine ethyl ester, Diamond: 8 mM Tyrosine ethyl ester with 1.25 uM Chymotrypsin, 20 min reaction/5 min SAM formation time.

FIG. 9 is a graph of the potential vs current when running a competitive enzymatic assay to detect N-benzoyl-L-tyrosine ethyl ester using an EAM with TAM and chymotrypsin. Differential signal can be seen for target concentrations. Line: 0 uM Tyrosine, Square: 31.25 uM Tyrosine, Asterisk: 125 uM Tyrosine, Circle: 500 uM Tyrosine, Diamond: 2 mM Tyrosine, Triangle: 8 mM Tyrosine with 312.5 nM Chymotrypsin, 20 min reaction/5 min SAM formation time.

FIG. 10 shows the EAM used in Examples 1-3, with TAM attached and detached. In this case, the TAM is a Tyrosine.

### DETAILED DESCRIPTION OF THE INVENTION

The present invention provides improved composition and methods for the detection and quantification of target analytes, such as small molecule target analytes, in the presence of an active enzyme by introducing an EAM that comprises a target analog moiety (TAM) that acts as a competitive substrate to the target analyte. In some embodiments, the EAM can be introduced in solution for a homogeneous reaction competing with the target analyte of interest and subsequently be detected after forming a self-assembled monolayer on an electrode. EAMs can be modified to attach substrates for enzymes (see for example US20140027310A1, such modified EAMs and related methods being incorporated herein by reference in their entirety) and still produce an output signal via other methodologies. Unexpectedly, such modified EAMs can provide suitable substrates to actually compete with natural targets for enzymatic activity. Thus, these molecules can be used to successfully create a new competitive enzymatic assay method for electrochemical detection of target analytes in a sample. Surprisingly, such EAMs modified to include TAMs react with enzymes in a consistent and measurable way. Significantly, these methods can allow for straightforward detection of otherwise very difficult targets to detect.

In conventional competition systems, detection is generally accomplished through displacement or binding or capture of the target. For example, conventional competition assays may utilize pre-tagged or labeled molecules that can bind to the same site as the target. Methods that focus capture may provide a number of binding sites that can be bound by either the target or an alternative labeled molecule. The target and the labeled molecule compete for binding sites, influenced by factors such as binding efficiency and concentration. Methods that focus on displacement may have either the target or a labeled molecule pre-bound to binding sites, with the other added after. Displacement occurs based on whether the target or the labeled molecules have a higher binding affinity, where those pre-bound may be “kicked out” by those added later if the binding affinity is higher. The binding or displacement can be measured through the detection of the label signaling molecules (e.g., florescent molecules) or reaction products (e.g., enzymatic reaction products) generated as a result of the target's presence.

The competition of the methods provided herein are quite different. The methods do not rely on the competition of the target analog moiety for binding sites. Additionally, the target itself is not directly involved in the generation of a signal. In fact, the target analyte is detected without a direct interaction. For example, in the methods, described herein, target analyte does not directly interact with the signaling molecule, does not participate in a binding step, is not captured by antibodies or on any surface, and a product formed from the enzymatic reaction utilizing the target analyte as a substrate is not measured or further used to generate a signal that can be measured. Further, the method of detection of the target analyte, describe herein, can be a label-free detection method, as it does not require any intermediary enzymes or surrogate targets. Rather, the signal is produced by the target analog moiety on the EAM.

The methods provided herein have a number of benefits. For example, the methods can be performed with fewer steps and reagents, and in a more straightforward manner. Another improvement of the compositions and methods of the present invention over conventional detection systems is that it expands the potential target molecules that may be detected by eliminating the requirement for a specific target ligand (e.g., antibody) and/or an enzymatic reaction product that can be utilized in some other chemical or enzymatic reaction to generate a detectable signal. In particular, it provides the ability to detect targets that are very difficult or otherwise impossible to detect without more complex detection technologies like mass spectrometry.

Creating detection schemes for target analytes utilizing binding interactions can be difficult because they often can only accommodate binding of one ligand, thereby limiting detection. Additionally developing binding ligands (e.g., antibodies) for target analytes can be much more challenging and expensive and often has sensitivity limitations due to lower affinity binding with the ligand. Moreover, it can be difficult to detect target analytes that don't produce reactive products from enzymatic reactions because it can be challenging or not possible to create a detectable change in a signal molecule because of the relative inertness of the reaction product, in some instances.

In some embodiments, the present invention provides for the quantification of a target analyte. The target may be measured on the basis of the concentration of reacted and unreacted EAMs using a competitive enzymatic assay format. For example, in some embodiments, with a finite amount of EAM added, the concentration of the reacted EAM is inversely correlated to the concentration of the unreacted EAM. In some embodiments, the amount of reacted EAM is inversely proportional to the target analyte concentration as both compete for the action of a finite amount of enzyme in a concentration-dependent manner. Thus, the ratio of reacted to unreacted EAM is accordingly inversely proportional to the target analyte concentration. In certain embodiments, even though this system relies on a competitive enzymatic assay format, there is no displacement of pre-formed complexes by the target. In some embodiments, the TAM is not displaced from the EAM by the introduction of the target analyte, but may be cleaved or modified by enzymatic activity. The TAM and the target analyte compete for enzymatic activity of an enzyme, for which both TAM and target analyte are substrates. The enzyme reacts with both the TAM and the target analyte at a rate which is concentration-dependent. If the starting concentration of EAM with TAM is held constant, the rate at which the enzyme will react with the TAM or the target becomes dependent upon the concentration of the target analyte in the sample. For example, if more target analyte is present, the enzyme will react with more of the target analyte and less of the EAM with the TAM attached. Accordingly, if less target analyte is present then the opposite will be true. Every enzymatic reaction with an EAM comprising a TAM contributes to a change in electrochemical signal which can be correlated to target analyte concentration. In some embodiments, EAM molecules have a first E⁰ when TAM is unreacted, and a second E⁰ when TAM is reacted such that the change in E^{o} can be measured and correlated to the amount of target analyte in the sample.

In one embodiment of any one of the methods or compositions provided herein, the sample is not exposed to enzyme before exposure to the EAM. In such an embodiment, the sample and the EAM containing TAM are exposed to the enzyme at the same time for competition to occur. Thus, in one embodiment of any one of the methods or compositions provided herein the sample is added to EAM and then the enzyme is added. In another embodiment of any one of the methods or compositions provided herein, the sample, enzyme and EAM are put in contact with each other at the same time.

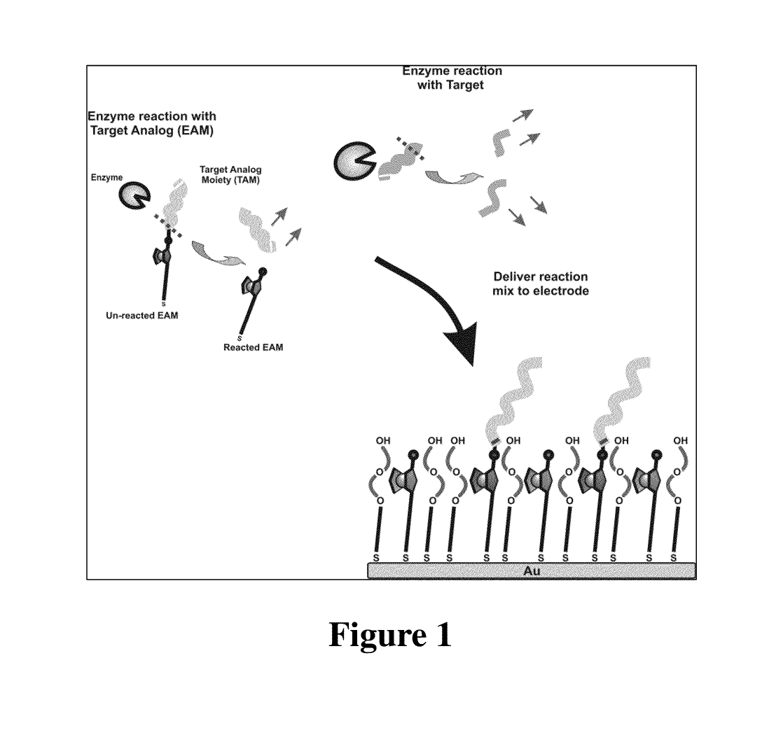

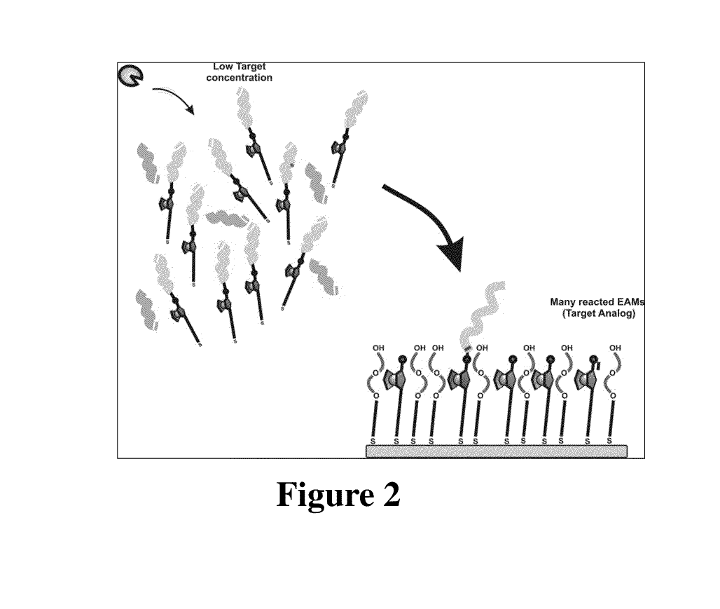

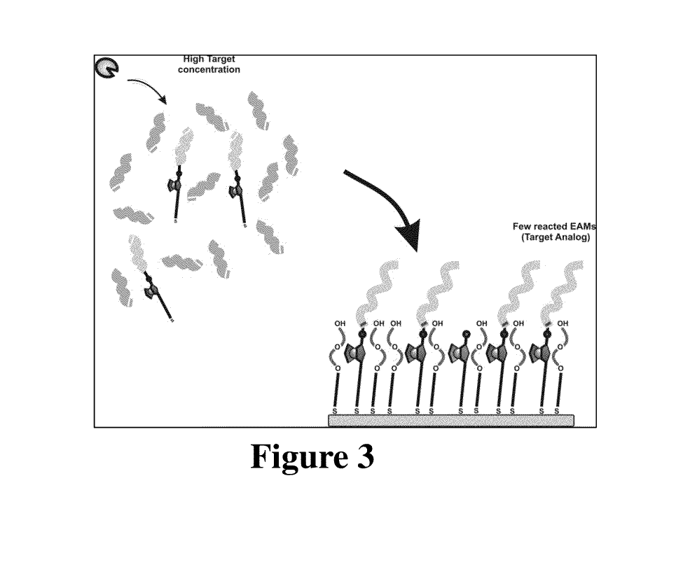

Non-limiting examples of the detection of small molecule target analytes using a competitive assay that utilizes an EAM comprising a TAM are illustrated in FIGS. 1-3. In some embodiments, as illustrated in FIGS. 1-3, the method is for determining at least one target analyte in a sample comprises exposing the sample to an EAM comprising a target analog moiety. The sample may also be exposed to an enzyme during and/or after exposure to the EAM. In some embodiments, the target analog moiety may have a similar or substantially the same activity for the enzyme as the target analyte. In some embodiments, the enzyme may react with at least a portion of the EAMs and/or target analyte, if present. In some embodiments, the ratio of reacted EAM to unreacted EAM after a certain incubation period with the enzyme and sample is dependent, at least in part, on the concentration of target analyte as well as the initial ratio of total EAM to target analyte (i.e., the ratio of EAM to target analyte before either has reacted with the enzyme). For example, as illustrated in FIG. 2, in embodiments in which the initial ratio of EAM to target analyte is relatively low (e.g., the concentration of the target analyte is greater than the concentration of the EAM), the concentration and/or relative percent of reacted EAM will be lower than the concentration and/or relative percent of target analyte that has reacted with enzyme. Conversely, e.g., as illustrated in FIG. 3, in embodiments in which the initial ratio of EAM to target analyte is relatively high (e.g., the concentration of the target analyte is less than the concentration of the EAM), the concentration and/or relative percent of reacted EAM will be higher than the concentration and/or relative percent of target analyte that has reacted with enzyme.

In some embodiments of any one of the methods or compositions provided herein, as illustrated in FIGS. 1-3, during and/or after exposing the EAM and target analyte to the enzyme, the resulting assay mixture may be brought in contact with a solid support. The solid support may comprise one or more electrodes. The EAMs from the assay mixture may self-assemble into a monolayer on the solid support. In some instances, the EAMs and/or solid support may comprise one or more moieties that facilitates self-assembly of the EAMs on the solid support. In certain embodiments, the ratio of reacted EAMs to unreacted EAMs on the solid support may be substantially the same as, similar to, or directly proportional to the ratio of reacted EAMs to unreacted EAMs in the assay mixture. In some such embodiments, ratio of reacted EAMs to unreacted EAMs may be determined from the self-assembled monolayer.

For instance, in some embodiments, the reacted EAMs and unreacted EAMs may have different E^{o}s. For example, the unreacted EAM may have a first E^{o} and the reacted EAM may have second E^{o} after at least a portion of the EAM (e.g., the TAM portion) is modified by the enzyme. That is, the EAM may have a first E^{o} when the TAM has not been modified by enzymatic reaction and a second E^{o} after the TAM has been modified by enzymatic reaction. An electrode may be used to determine the proportion of the second E^{o} to the first E^{o} to determine the ratio of reacted EAMs to unreacted EAMs. Holding constant the amount of enzyme and EAM used, the concentration of target analyte in the sample is the variable that most heavily determines whether the enzyme will react with the target or the TAM of the EAM. The TAM attached on the EAM and the target analyte are competitive substrates for an enzyme, which reacts with both of the above substrates in a rate-dependent manner. Holding the enzyme and EAM concentration constant, the rate becomes dependent upon the concentration of the target analyte in the sample. For example, if more target analyte is present, more target analyte will be reacted and less of the TAM, which is part of the electro-active moiety, will be catalyzed by or otherwise react with the enzyme. Hence, this will result in a higher number of unreacted EAMs (where TAM is not modified) than the number of reacted EAM (where TAM is modified). Accordingly, if less target analyte is present, then less of the target analyte will be acted upon by the enzyme and more of the TAM will be catalyzed. Hence, this will result in a lesser number of unreacted EAMs than the number of reacted EAM.

After the enzymatic reaction with the TAM and target analyte either in a solution phase or surface based assay, the unreacted EAM (having a first E^{o}) and the reacted EAM (having a second E^{o}) generate a measurable signal which can be used to determine the concentration of target analyte in the sample. In some embodiments of any one of the methods or compositions provided herein, the ratio of the signal of reacted EAM to the signal of unreacted EAM, i.e., a ratio of the second E^{o} to the first E^{o}, is used. This ratio of second E^{o} to first E^{o} is inversely correlated to the concentration of the target analyte in the test sample.

In one example of any one of the methods provided herein, the method may comprise a solution phase assay wherein the test sample containing the target analyte is contacted with the EAM comprising a TAM and a transition metal complex, said EAM having a first E^{o} when said TAM is unreacted (i.e., has not been modified by enzymatic activity) and a second E^{o} when said TAM is reacted (i.e., has been enzymatically modified), along with an enzyme for which both the target analyte and TAM are substrates, in solution to form an assay mix. In some embodiments of any one of the methods provided herein, these contacting steps may be done in sequence, while in other embodiments of any one of the methods provided herein they may be done simultaneously. Generally, the enzyme reacts with both the TAM and the target analyte at a constant rate. If known enzyme and EAM concentrations are used, the rate at which the enzyme acts on the EAM and the target becomes dependent upon the concentration of the target analyte in the sample. For example, if more target analyte is present, the enzyme is more likely to encounter the target analyte in solution than the TAM. Thus more target analyte will be reacted, and less of the TAM will be catalyzed by or otherwise react with the enzyme. Accordingly, if less target analyte is present then the opposite will be true. This solution phase assay mixture can then be contacted with a solid support comprising an electrode, where both the reacted and unreacted EAMs self-assemble to form a monolayer. The electrode is interrogated and signal is measured. In some embodiments of any one of the methods provided herein, cyclic voltammetry can be used to detect electrochemical potentials of unreacted and reacted EAM. The ratio of the reacted EAM (second E^{o}) to the unreacted EAM (first E^{o}) is calculated, which is inversely proportional to the concentration of target analyte in the sample.

In another embodiment of any one of the methods or compositions provided herein, the method may comprise a surface based assay wherein the EAM comprising TAM and a transition metal complex, said EAM having a first E^{o} when said TAM is unreacted (i.e. has not been modified by enzymatic activity) and a second E^{o} when said TAM is reacted (i.e. has been enzymatically modified), is covalently attached to a solid support comprising an electrode to form a pre-formed self-assembled monolayer. In some embodiments of any one of the methods or compositions provided herein, the self-assembled monolayer may also contain a diluent species. The test sample containing the target analyte can then be contacted with the electrode surface and consequently, contacts the EAM in the monolayer. An enzyme, for which both the target analyte and TAM are substrates, is also added. The enzyme can react with both the TAM and the target analyte at a constant rate. If known enzyme and EAM concentrations are used, the rate at which the enzyme acts on the EAM and the target becomes dependent upon the concentration of the target analyte in the sample. For example, if more target analyte is present, the enzyme is more likely to encounter the target analyte in solution than the TAM. Thus more target analyte will be reacted, and less of the TAM will be catalyzed by or otherwise react with the enzyme. Accordingly, if less target analyte is present then the opposite will be true. Once the reacting has taken place, the signal can be measured via the electrode and used to determine target analyte concentration. In some embodiments of any one of the methods provided herein, cyclic voltammetry is used to detect electrochemical potentials of unreacted and reacted EAM. In some embodiments of any one of the methods provided herein, the ratio of the reacted EAM (second E^{o}) to the unreacted EAM (first E^{o}) is calculated, which is inversely proportional to the concentration of target analyte in the sample.

In one embodiment of any one of the methods provided herein, the compositions of the invention are added either simultaneously or sequentially in the assay, either in the solution phase assay mixture or on the solid support (electrode). That is, in one embodiment of any one of the methods provided herein, the test sample and the enzyme, for which the target and target analog moiety are substrates, are contacted with the EAM simultaneously in the solution phase assay mixture or in the surface based assay. In one embodiment of any one of the methods provided herein, the components are added sequentially: first the test sample is contacted with the EAM to form a first assay mixture, followed by contacting the first assay mixture with the enzyme to form a second assay mixture.

As will be understood by those in the art, additional assay components and process aids can be added and varied to provide optimal conditions for this reaction method (e.g. a buffer that provides an ideal pH for enzymatic function).

In general, any suitable EAM may be used. As described herein, the composition of the EAMs used in certain embodiments of the invention includes an analog of the target analyte, which is similar in structure and function to the target attached to the transition metal complex. In some embodiments of any one of the methods or compositions provided, the EAM may be configured such that the target analog moiety is a functional substrate to the enzyme. In a preferred embodiment of any one of the methods or compositions provided, the TAM of the EAM exhibits similar enzymatic activity as the native target analyte to enzyme. In some embodiments of any one of the methods or compositions provided, the EAM has some distinguishing electrochemical or self-assembling characteristic that allows for a detectable change once the target analog moiety is modified. TAMs can be enzymatically modified in multiple ways, including but not limited to adding an additional functional group, removing a functional group, altering the chemical structure, and cleaving the TAM off of the EAM.

In one aspect, the invention provides compositions and methods for detecting at least one target analyte in a test sample, said method comprising:contacting a test sample with an electroactive moiety (EAM) and at least one enzyme, for which the target and target analog moiety are substrates, said EAM comprising a transition metal complex and an target analog moiety (TAM) and having a first E^{o}, under conditions wherein said TAM is modified (removed/restructured) from at least a portion of said EAM by said at least one enzyme and results in said EAM having a second E^{o}; detecting for a change between the first E^{o} and the second E^{o} of said EAM, wherein said change is an indication of the presence of said at least one target analyte.

As used herein, EAM, Target analog EAM, EAM with TAM, target analog molecule, or equivalent is an electro-active moiety (EAM), comprised of a transition metal complex, anchor group, target analog moiety (TAM) and optionally linker or self-immolative linker groups.

As used herein, target analog moiety (TAM) is a group that has a similar or analogous structure and function to the target of interest that may include a linker or other functional group that serves to attach the TAM to the EAM such that when the TAM is modified (removed/restructured) the EAM exhibits distinguishable electrochemical or self-assembling characteristics from the original, unreacted target analog EAM.

Distinguishable electrochemical or self-assembling characteristics include shift in redox potential, entirely new redox potential, change in current measured at certain redox potential, change in rate or efficiency of self-assembling into a monolayer, such as on gold.

In one embodiment of any one of the methods or compositions provided, the assay mixture is in a solution phase. For example the assay mixture may be formed in step (a) and prior to step (b), such a method further comprising:

(a1) contacting said assay mixture with a solid support comprising an electrode or an array of electrodes, under conditions such that a self-assembled monolayer (SAM) forms on said electrode, said EAM having said first E^{o} and said EAM having said second E^{o}. Thus, in one embodiment of this aspect provided is a method for detecting at least one target analyte in a test sample, said method comprising:

(a) contacting a test sample with an electroactive moiety (EAM) and at least one at least one enzyme, for which the target and target analog moiety are substrates, to form an assay mixture in solution phase, said EAM having a first E^{o} and comprising a transition metal complex and an target analog moiety (TAM), under conditions wherein said TAM is modified (removed/restructured) from at least a portion of said EAM by said at least one enzyme resulting in said EAM having a second E^{o};

(b) contacting said assay mixture with a solid support comprising an electrode or an array of electrodes under conditions such that a self-assembled monolayer (SAM) forms on said electrode, said EAM having said first E^{o} and said EAM having said second E^{o}; and

(c) detecting for a change between the first E^{o} and the second E^{o} of said EAM, wherein said change is an indication of the presence of said at least one target analyte.

In one embodiment of any one of the methods or compositions provided, said EAM is covalently attached to an electrode or array of electrodes on a solid support as a self-assembled monolayer (SAM). In one embodiment provided is a method for detecting at least one target analyte in a test sample, said method comprising:
(a) contacting a test sample with a solid support, said solid support comprising an electrode or array of electrodes, said electrode comprising:
   (i) a self-assembled monolayer; and
   (ii) a covalently attached electroactive moiety (EAM), said EAM having a first E^{o} and comprising a transition metal complex and an target analog moiety (TAM),

under conditions wherein said TAM is modified (removed/restructured) from at least a portion of said EAM by at least one enzyme, for which the target and target analog moiety are substrates, and results in said EAM having a second E^{o}; and
(b) detecting a change between the first E^{o} and the second E^{o} of said EAM, wherein said change is an indication of the presence of said at least one target analyte.

In another embodiment of any one of the methods provided herein, the compositions of the invention are added either simultaneously or sequentially in the assay, either in the solution phase assay mixture or on the solid support (electrode). That is, in one embodiment of any one of the methods provided, the test sample and the enzyme, for which the target and target analog moiety are substrates, are contacted with the EAM simultaneously in the solution phase assay mixture or in the surface based assay. In one embodiment of any one of the methods provided, the components can be added sequentially; first the test sample is contacted with the EAM followed by contacting both with the enzyme. Thus, in one embodiment provided is a method for detecting at least one target analyte in a test sample, said method comprising:
(a) contacting a test sample with an electroactive moiety (EAM) to form an assay mixture in solution phase, said EAM having a first E^{o} and comprising a transition metal complex and an target analog moiety (TAM) to form a first assay mixture;
(b) contacting the first assay mixture and at least one at least one enzyme for which the target and target analog moiety are substrates in solution phase to form a second assay mixture, under conditions wherein said TAM is modified (removed/restructured) from at least a portion of said EAM by said at least one enzyme resulting in said EAM having a second E^{o};
(c) contacting said second assay mixture with a solid support comprising an electrode or an array of electrodes under conditions such that a self-assembled monolayer (SAM) forms on said electrode, said EAM having said first E^{o} and said EAM having said second E^{o}; and
(d) detecting for a change between the first E^{o} and the second E^{o} of said EAM, wherein said change is an indication of the presence of said at least one target analyte

In one embodiment of any one of the methods or compositions provided the EAM further comprises a self-immolative moiety (SIM) which joins said TAM to said transition metal complex.

In one embodiment of any one of the methods or compositions provided said target analog moiety (TAM) is selected from the group consisting of amino acids, peptides, nucleic acids, metabolites, neurotransmitters, acetate, lipids, fatty acids, glycolipids, phospholipids, sphingolipids, saccharides, polysaccharides, oligomers, phosphates, steroids, hormones, vitamins, or other functional group.

In one embodiment of any one of the methods or compositions provided said at least one target analyte is selected from the group consisting of amino acids, peptides, nucleic acids, metabolites, neurotransmitters, acetate, lipids, fatty acids, glycolipids, phospholipids, sphingolipids, saccharides, polysaccharides, oligomers, phosphates, steroids, hormones, vitamins, or other functional group. In another embodiment of any one of the methods or compositions provided said at least one target analyte is a small molecule.

In one embodiment of any one of the methods or compositions provided said at least one enzyme, for which the target and target analog moiety are substrates, is selected from the group consisting of proteases, peptidases, phosphatases, oxidases, hydrolases, lyases, transferases, isomerase, ligases, and other enzymes that remove a functional group from a substrate or co-substrate.

In one embodiment of any one of the methods or compositions provided said transition metal complex includes a transition metal selected from the group consisting of iron, ruthenium and osmium.

In one embodiment of any one of the methods or compositions provided said transition metal complex comprises a ferrocene or substituted ferrocene.

In one embodiment of any one of the methods or compositions provided said EAM comprises a flexible oligomer anchor tethering said transition metal complex to said electrode, said flexible anchor being an oligomers with polar or charged functional groups in their main chain or side chains. Examples include poly acrylic acids, polyethylene glycol (PEG), poly vinyl alcohol, polymethacrylate, poly vinylpyrrolidinone, acrylamide, maleic anhydride, and poly vinylpyridine, allylamine, ethyleneimine, oxazoline, and other hydrophobic oligomers with side chains that limit intermolecular hydrophobic interactions and prevent organization and rigidity.

In one embodiment of any one of the methods or compositions provided each electrode in said array of electrodes is modified with a SAM comprising a unique EAM, each EAM comprising a unique TAM for a specific target analyte such that two or more different target analytes may be detected in said test sample when two or more enzymes, which are each selective for respective target/target analog pairs, are introduced.

Another aspect of the disclosure provides compositions comprising a solid support comprising an electrode comprising:
(i) a self-assembled monolayer (SAM); and
(ii) a covalently attached electroactive active moiety (EAM) comprising a transition metal complex and an target analog moiety (TAM), wherein said EAM has a first E⁰ when said TAM is present and a second E⁰ when said TAM is absent.

In one embodiment of any one of the methods or compositions provided, the EAM further comprises a self-immolative moiety (SIM) that joins the TAM to the transition metal complex.

Another aspect of the disclosure provides a kit for detecting at least one target analyte in a test sample, the kit comprising any one of the compositions provided herein.

### Target Analytes

By “target analyte” or “analyte” or “target” or grammatical equivalents herein is meant any molecule, compound, or particle to be detected. Basically, any molecule, which can be reacted upon by an enzyme and for which an analog is available, can be detected as the target in this invention. Target analytes which are too small to be detected by antibodies find particular use in this invention. Suitable target analytes include but are not limited to, amino acids, peptides, nucleic acids, metabolites, neurotransmitters, acetate, lipids, fatty acids, glycolipids, phospholipids, sphingolipids, saccharides, polysaccharides, oligomers, phosphates, steroids, hormones, vitamins, and other functional groups. In one embodiment of any one of the methods or compositions provided, the analyte may be an environmental pollutant (including pesticides, insecticides, toxins, etc.); a chemical (including solvents, polymers, organic materials, etc.); therapeutic molecule (including therapeutic and abused drugs, antibiotics, etc.) or biomolecule; etc.

### Small Molecule

As used herein, the term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments of any one of the methods or compositions provided, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible. In certain embodiments of any one of the methods or compositions provided, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions.

### Test Samples

The target analytes are generally present in samples. As will be appreciated by those in the art, the sample solution may comprise any number of things, including, but not limited to, bodily fluids (including, but not limited to, blood, urine, serum, lymph, saliva, anal and vaginal secretions, perspiration, tears, prostatic fluid, and semen samples of virtually any organism, with mammalian samples being preferred and human samples being particularly preferred); environmental samples (including, but not limited to, air, agricultural, water and soil samples); plant materials; biological warfare agent samples; research samples; purified samples; raw samples; etc. As will be appreciated by those in the art, virtually any experimental manipulation and/or sample preparation may have been done on the sample. Some embodiments utilize target samples from stored (e.g. frozen and/or archived) or fresh tissues. Paraffin-embedded samples are of particular use in some embodiments, as these samples can be very useful due to the presence of additional data associated with the samples, such as diagnosis and prognosis. Fixed and paraffin-embedded tissue samples as described herein refers to storable or archival tissue samples. Most patient-derived pathological samples are routinely fixed and paraffin-embedded to allow for histological analysis and subsequent archival storage.

### Target Analog Moiety (TAM)

Target analog moiety (TAM) or electro-active target analog is a group that has a similar, analogous or identical structure, function to the target of interest and is a substrate analog of the target. It may include a linker or other functional group that serves to attach the TAM to the EAM such that when the TAM is modified (removed/restructured) the EAM exhibits distinguishable electrochemical or self-assembling characteristics from the original, unreacted target analog EAM. Target analog moiety (TAM) can be any molecule which is structurally, functionally and chemically similar or identical to the target. In general TAM can be selected from any of the following groups, including but not limited to, amino acids, peptides, nucleic acids, metabolites, neurotransmitters, acetate, lipids, fatty acids, glycolipids, phospholipids, sphingolipids, saccharides, polysaccharides, oligomers, phosphates, steroids, hormones, vitamins, or other functional group. In a preferred embodiment of any one of the methods or compositions provided, the structure, sequence and chemistry of the site of enzymatic interaction of the TAM is similar to, substantially similar to, or identical to the site of enzymatic interaction of the target of interest and exhibits similar, substantially similar, or identical enzymatic kinetics with the enzyme as the target.

As will be understood by those in the art, suitable target analog molecules can be chosen and attached to EAMs via conventional synthetic means. In some embodiments of any one of the methods or compositions provided, the TAM may be attached to the EAM using suitable conjugation chemistry that utilizes functional groups, which do not participate in the enzymatic reaction. For example, chymotrypsin is known to act on amide bonds when the side chain contains aromatic components (e.g. tyrosine, phenylalanine, and tryptophan). This suggests that an amino acid such as tyrosine would make a suitable TAM when coupled to an EAM molecule via an amide bond. Such an EAM with tyrosine TAM can be synthesized according to normal synthetic processes. See Example 4 for detailed procedure of one such method that can be used.

Testosterone is an example target wherein aromatase (or 5-alpha reductase or 3alpha-hydroxysteroid 3-dehydrogenase) could competitively utilize testosterone and a testosterone analog EAM as substrates producing a measurable signal output dependent on the concentration of testosterone in a sample. This detection scheme is advantageous for the detection of testosterone because the product formed by aromatase (or 5-alpha reductase or 3alpha-hydroxysteroid 3-dehydrogenase) are androgens or estrogen hormone derivatives that are not readily exploitable for signal generation and/or detection.

Epinephrine is another example of a target which does not act as a substrate for an enzyme that produces a reaction product suitable for use in a detection scheme. This Competitive Enzymatic Assay however could allow for epinephrine detection by utilizing catechol O-methyltransferase an enzyme which would use both epinephrine and the target analog EAM (epinephrine-EAM) as substrates.

The targets analytes that could be detected with this invention include small molecules that act as a substrate for an enzyme.

### Enzyme

Generally, the present invention provides for detection and quantification of the target analyte, through a competitive enzymatic assay format by utilizing an enzyme which reacts with both the target and the TAM in a concentration dependent manner, e.g., if more target is present, more target will be reacted and less electro-active target analog moiety will be reacted; if less target is present then the opposite will be true. The enzyme, for which the target and target analog moiety are substrates, can be selected from the following groups, including but not limited to, proteases, peptidases, phosphatases, oxidases, hydrolases, lyases, transferases, isomerase, ligases, or other enzyme that modifies (removes/restructures) a functional group from a substrate or co-substrate.

As described herein, the enzyme may modify at least a portion of the EAM. For example, the enzyme may modify at least a portion of the TAM. In such cases, the enzyme may cause the TAM to undergo a chemical reaction the at least temporarily alters the chemical structure of the TAM and/or removes the TAM from the EAM. In general, modification of the TAM may result in a detectable change in the E^{o} of the EAM.

### Solid Support

The target analytes can be detected using solid supports comprising electrodes. By “substrate” or “solid support” or other grammatical equivalents herein is meant any material that can be modified to contain discrete individual sites appropriate for the attachment or association of SAMs or EAMs. Suitable substrates include metal surfaces such as gold, electrodes as defined below, glass and modified or functionalized glass, fiberglass, Teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc., polysaccharides, nylon or nitrocellulose, resins, silica or silica-based materials including silicon and modified silicon, carbon, metals, inorganic glasses and a variety of other polymers, with evaporated gold circuits on a polymer backing, etc.

The present system finds particular utility in array formats, i.e. wherein there is a matrix of addressable detection electrodes (herein generally referred to “pads”, “addresses” or “micro-locations”). By “array” herein is meant an array of electrodes, with each electrode modified with a SAM comprising a unique EAM, each EAM comprising a unique TAM for a specific target analyte such that two or more different target analytes may be detected in said test sample in some embodiments.

In a preferred embodiment of any one of the methods or compositions provided herein, the detection electrodes are formed on a substrate. In addition, the discussion herein is generally directed to the use of gold electrodes, but as will be appreciated by those in the art, other electrodes can be used as well. The substrate can comprise a wide variety of materials, as outlined herein and in the cited references, the disclosures of such materials of which are herein incorporated by reference in their entirety.

In general, materials include printed circuit board materials. Circuit board materials are those that generally comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g., all electrodes and interconnections in a plane) or “three dimensional” (wherein the electrodes are on one surface and the interconnects may go through the board to the other side or wherein electrodes are on a plurality of surfaces) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer. Accordingly, in a preferred embodiment, the present invention provides chips that comprise substrates comprising a plurality of electrodes, preferably gold electrodes. The number of electrodes is as outlined for arrays. Each electrode can become modified with a self-assembled monolayer in situ during the last step of the assay as outlined herein. In addition, each electrode can have an interconnection, that is the electrode is ultimately attached to a device that can control the electrode. That is, each electrode can be independently addressable.

Finally, the compositions of the invention can include a wide variety of additional components, including microfluidic components and robotic components (see for example U.S. Pat. Nos. 6,942,771 and 7,312,087 and related cases, the disclosures of such components of both of which are hereby incorporated by reference in their entirety), and detection systems including computers utilizing signal processing techniques (see for example U.S. Pat. No. 6,740,518, the disclosures of such systems being herein incorporated by reference in their entirety).

### Self-Assembled Monolayers

The electrodes can comprise either a pre-formed self-assembled monolayer (SAM) or a SAM formed in situ as part of the homogenous assay. By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules can include a functional group that adheres to the surface, and a portion that interacts with neighboring molecules in the monolayer to form the relatively ordered array. A “mixed” monolayer comprises a heterogeneous monolayer, that is, where at least two different molecules make up the monolayer. As outlined herein, the use of a monolayer can reduce the amount of non-specific binding of biomolecules to the surface, and, in the case of nucleic acids, increases the efficiency of oligonucleotide hybridization as a result of the distance of the oligonucleotide from the electrode. In addition, a monolayer can serve to keep charge carriers away from the surface of the electrode.

In some embodiments the monolayer comprises oligomers, and in particular, oligomers are generally used to attach the EAM to the electrode surface, as described below. In a preferred embodiment the oligomers are flexible and have limited interaction with adjacent molecules such that there is little if any rigidity or organization. Additionally these oligomers may be hydrophilic in order to present a more accessible interface for enzymatic interaction. Due to the disorder and flexibility, these oligomers need not be conductive as the transition metal complex is near enough with sufficient access to the electrode surface as well as the supporting counter ion electrolyte for direct electronic communication through solution to the electrode. Preferred flexible hydrophilic oligomers include oligomers with polar or charged functional groups in their main chain or side chains with these characteristics. Hydrophilic oligomers are also preferred in some embodiments because they increase the solubility of the EAM in aqueous samples. Aqueous samples are ideal, in some embodiments, for highest enzyme activity therefore EAMs that are more aqueous soluble require less organic solvent to perform the target EAM reaction which in turn will yield higher signal due to increased enzymatic activity. Examples include poly acrylic acids, polyethylene glycol (PEG), poly vinyl alcohol, polymethacrylate, poly vinylpyrrolidinone, acrylamide, maleic anhydride, and poly vinylpyridine. Amine functional oligomers could also be used including allylamine, ethyleneimine, and oxazoline. Other hydrophobic oligomers could be used as well, in particular, oligomers with side chains that limit intermolecular hydrophobic interactions and therefore prevent organization and rigidity. Hydrophobic oligomer linkers could be better suited to particular enzymes as they may have more favorable interactions with hydrophobic regions near enzyme active sites. Ideal oligomer lengths may depend on the target enzyme and monomer structure, with longer oligomers being optimal for enzymatic access but with upper length limitations imposed by electrochemical performance.

In some embodiments, the monolayer comprises conductive oligomers, and in particular, conductive oligomers are generally used to attach the EAM to the electrode surface, as described below. By “conductive oligomer” herein is meant a substantially conducting oligomer, preferably linear, some embodiments of which are referred to in the literature as “molecular wires”. By “substantially conducting” herein is meant that the oligomer is capable of transferring electrons at 100 Hz. Generally, the conductive oligomer has substantially overlapping π-orbitals, i.e., conjugated π-orbitals, as between the monomeric units of the conductive oligomer, although the conductive oligomer may also contain one or more sigma (σ) bonds. Additionally, a conductive oligomer may be defined functionally by its ability to inject or receive electrons into or from an associated EAM. Furthermore, the conductive oligomer is more conductive than the insulators as defined herein. Additionally, the conductive oligomers of the invention are to be distinguished from electro-active polymers, that themselves may donate or accept electrons.

A more detailed description of conductive oligomers is found in WO/1999/57317, such description being herein incorporated by reference in its entirety. In particular, the conductive oligomers as shown in Structures 1 to 9 on page 14 to 21 of WO/1999/57317 find use in the present invention in some embodiments. In some embodiments, the conductive oligomer has the following structure:

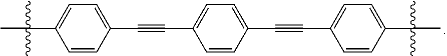 [SMILES: *c1ccc(C#Cc2ccc(C#Cc3ccc(*)cc3)cc2)cc1]

In addition, the terminus of at least some of the conductive oligomers in the monolayer can be electronically exposed. By “electronically exposed” herein is meant that upon the placement of an EAM in close proximity to the terminus, and after initiation with the appropriate signal, a signal dependent on the presence of the EAM may be detected. The conductive oligomers may or may not have terminal groups. Thus, there may be no additional terminal group, and the conductive oligomer terminates with a terminal group; for example, such as an acetylene bond. Alternatively, in some embodiments, a terminal group is added, sometimes depicted herein as “Q”. A terminal group may be used for several reasons; for example, to contribute to the electronic availability of the conductive oligomer for detection of EAMs, or to alter the surface of the SAM for other reasons, for example to prevent non-specific binding. For example, there may be negatively charged groups on the terminus to form a negatively charged surface such that when the target analyte is nucleic acid such as DNA or RNA, the nucleic acid is repelled or prevented from lying down on the surface, to facilitate hybridization. Preferred terminal groups include —NH, —OH, —COOH, and alkyl groups such as —CH₃, and (poly)alkyloxides such as (poly)ethylene glycol, with —OCH₂CH₂OH, —(OCH₂CH₂O)₂H, —(OCH₂CH₂O)₃H, and —(OCH₂CH₂O)₄H being preferred.

In one embodiment, it is possible to use mixtures of conductive oligomers with different types of terminal groups. Thus, for example, some of the terminal groups may facilitate detection, and some may prevent non-specific binding.

Passivation agents can serve as a physical barrier to block solvent accessibility to the electrode. As such, the passivation agents themselves may in fact be either (1) conducting or (2) nonconducting, i.e. insulating, molecules. Thus, in one embodiment, the passivation agents are conductive oligomers, as described herein, with or without a terminal group to block or decrease the transfer of charge to the electrode. Other passivation agents which may be conductive include oligomers of —(CF₂)ₙ—, —(CHF)ₙ— and —(CFR)ₙ—. In a preferred embodiment, the passivation agents are insulator moieties.

In some embodiments, the monolayers comprise insulators. An “insulator” is a substantially nonconducting oligomer, preferably linear. By “substantially nonconducting” herein is meant that the rate of electron transfer through the insulator is slower than the rate of electron transfer through the conductive oligomer. Stated differently, the electrical resistance of the insulator is higher than the electrical resistance of the conductive oligomer. It should be noted however that even oligomers generally considered to be insulators, such as —(CH₂)₁₆ molecules, still may transfer electrons, albeit at a slow rate.

In some embodiments, the insulators have a conductivity, S, of about 10⁻⁷ Ω-1 cm⁻¹ or lower, with less than about 10⁻⁸ Ω⁻¹cm⁻¹ being preferred. Gardner et al., Sensors and Actuators A 51 (1995) 57-66, incorporated herein by reference.

Generally, insulators are alkyl or heteroalkyl oligomers or moieties with sigma bonds, although any particular insulator molecule may contain aromatic groups or one or more conjugated bonds. By “heteroalkyl” herein is meant an alkyl group that has at least one heteroatom, i.e. nitrogen, oxygen, sulfur, phosphorus, silicon or boron included in the chain. Alternatively, the insulator may be quite similar to a conductive oligomer with the addition of one or more heteroatoms or bonds that serve to inhibit or slow, preferably substantially, electron transfer. In some embodiments the insulator comprises C₆-C₁₆ alkyl.

The passivation agents, including insulators, may be substituted with R groups as defined herein to alter the packing of the moieties or conductive oligomers on an electrode, the hydrophilicity or hydrophobicity of the insulator, and the flexibility, i.e. the rotational, torsional or longitudinal flexibility of the insulator. For example, branched alkyl groups may be used. In addition, the terminus of the passivation agent, including insulators, may contain an additional group to influence the exposed surface of the monolayer, sometimes referred to herein as a terminal group (“TG”). For example, the addition of charged, neutral or hydrophobic groups may be done to inhibit non-specific binding from the sample, or to influence the kinetics of binding of the analyte, etc. For example, there may be charged groups on the terminus to form a charged surface to encourage or discourage binding of certain target analytes or to repel or prevent from lying down on the surface.

The length of the passivation agent may vary as needed. Generally, the length of the passivation agents is similar to the length of the conductive oligomers, as outlined above. In addition, the conductive oligomers may be basically the same length as the passivation agents or longer than them.

The in situ monolayer may comprise a single type of passivation agent, including insulators, or different types.

Suitable insulators are known in the art, and include, but are not limited to, —(CH₂)ₙ—, —(CRH)ₙ—, and —(CR₂)ₙ—, ethylene glycol or derivatives using other heteroatoms in place of oxygen, i.e. nitrogen or sulfur (sulfur derivatives are not preferred when the electrode is gold). In some embodiments, the insulator comprises C6 to C16 alkyl.

In some embodiments, the electrode is a metal surface and need not necessarily have interconnects or the ability to do electrochemistry.

### Electro-Active Moieties (EAM)

In addition to the SAMs, the in situ modified electrodes comprise an EAM in some embodiments. By “electroactive moiety (EAM)” or “transition metal complex” or “redox active molecule” or “electron transfer moiety (ETM)” herein is meant a metal-containing compound which is capable of reversibly or semi-reversibly transferring one or more electrons. It is to be understood that electron donor and acceptor capabilities are relative; that is, a molecule which can lose an electron under certain experimental conditions may be able to accept an electron under different experimental conditions.

It is to be understood that the number of possible transition metal complexes is very large, and that one skilled in the art of electron transfer compounds will be able to utilize a number of compounds in the present invention. By “transitional metal” herein is meant metals whose atoms have a partial or completed shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, find particular use in the present invention. Metals that find use in the invention also are those that do not change the number of coordination sites upon a change in oxidation state, including ruthenium, osmium, iron, platinium and palladium, with osmium, ruthenium and iron being especially useful. Generally, transition metals are depicted herein (or in incorporated references) as TM or M.

The transitional metal and the coordinating ligands form a metal complex. By “ligand” or “coordinating ligand” (depicted herein or in incorporated references in the figures as “L”) herein is meant an atom, ion, molecule, or functional group that generally donates one or more of its electrons through a coordinate covalent bond to, or shares its electrons through a covalent bond with, one or more central atoms or ions.

In some embodiments, small polar ligands are used; suitable small polar ligands, generally depicted herein as “L”, fall into two general categories, as is more fully described herein. In one embodiment, the small polar ligands will be effectively irreversibly bound to the metal ion, due to their characteristics as generally poor leaving groups or as good sigma donors, and the identity of the metal. These ligands may be referred to as “substitutionally inert”. Alternatively, as is more fully described below, the small polar ligands may be reversibly bound to the metal ion, such that upon binding of a target analyte, the analyte may provide one or more coordination atoms for the metal, effectively replacing the small polar ligands, due to their good leaving group properties or poor sigma donor properties. These ligands may be referred to as “substitutionally labile”. The ligands preferably form dipoles, since this can contribute to a high solvent reorganization energy.

Some of the structures of exemplary transitional metal complexes are shown below:

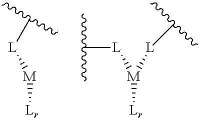

L are the co-ligands, that provide the coordination atoms for the binding of the metal ion. As will be appreciated by those in the art, the number and nature of the co-ligands will depend on the coordination number of the metal ion. Mono-, di- or polydentate co-ligands may be used at any position. Thus, for example, when the metal has a coordination number of six, the L from the terminus of the conductive oligomer, the L contributed from the nucleic acid, and r, add up to six. Thus, when the metal has a coordination number of six, r may range from zero (when all coordination atoms are provided by the other two ligands) to four, when all the co-ligands are monodentate. Thus generally, r will be from 0 to 8, depending on the coordination number of the metal ion and the choice of the other ligands.

In one embodiment, the metal ion has a coordination number of six and both the ligand attached to the conductive oligomer and the ligand attached to the nucleic acid are at least bidentate; that is, r is preferably zero, one (i.e. the remaining co-ligand is bidentate) or two (two monodentate co-ligands are used).

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lm). Suitable nitrogen donating ligands are well known in the art and include, but are not limited to, cyano (C≡N), NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp 73-98), 21.1 (pp. 813-898) and 21.3 (pp 915-957), all of which are hereby expressly incorporated by reference.

As will be appreciated in the art, any ligand donor (1)-bridge-donor (2) where donor (1) binds to the metal and donor (2) is available for interaction with the surrounding medium (solvent, protein, etc) can be used in the present invention, especially if donor (1) and donor (2) are coupled through a pi system, as in cyanos (C is donor (1), N is donor (2), pi system is the CN triple bond). One example is bipyrimidine, which looks much like bipyridine but has N donors on the “back side” for interactions with the medium. Additional co-ligands include, but are not limited to, cyanates, isocyanates (—N═C═O), thiocyanates, isonitrile, N₂, O₂, carbonyl, halides, alkoxyide, thiolates, amides, phosphides, and sulfur containing compound such as sulfino, sulfonyl, sulfoamino, and sulfamoyl.

In some embodiments, multiple cyanos are used as co-ligand to complex with different metals. For example, seven cyanos bind Re(III); eight bind Mo(IV) and W(IV). Thus at Re(III) with 6 or less cyanos and one or more L, or Mo(IV) or W(IV) with 7 or less cyanos and one or more L can be used in the present invention. The EAM with W(IV) system has particular advantages over the others in some embodiments because it is more inert, easier to prepare, more favorable reduction potential. Generally a larger CN/L ratio will give larger shifts.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands can be attached in such a manner as to allow the heteroatoms to serve as coordination atoms.

In some embodiments, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with .pi.-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C5H5 (−1)] and various ring substituted and ring fused derivatives, such as the indenylide (−1) ion, that yield a class of bis(cyclopentadieyl)metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example. Other acyclic π-bonded ligands such as the allyl(−1) ion, or butadiene yield potentially suitable organometallic compounds, and all such ligands, in conduction with other .pi.-bonded and .delta.-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra), in some embodiments. For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

As a general rule, EAM comprising non-macrocyclic chelators can be bound to metal ions to form non-macrocyclic chelate compounds, since the presence of the metal allows for multiple proligands to bind together to give multiple oxidation states.

In some embodiments, nitrogen donating proligands are used. Suitable nitrogen donating proligands are well known in the art and include, but are not limited to, NH2; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. It should be noted that macrocylic ligands that do not coordinatively saturate the metal ion, and which require the addition of another proligand, are considered non-macrocyclic for this purpose. As will be appreciated by those in the art, it is possible to covalent attach a number of “non-macrocyclic” ligands to form a coordinatively saturated compound, but that is lacking a cyclic skeleton.

In some embodiments, a mixture of monodentate (e.g., at least one cyano ligand), bi-dentate, tri-dentate, and polydentate ligands can be used in the construction of EAMs.

Of particular use in the present invention are EAMs that are metallocenes, and in particular ferrocenes, which have at least a first self-immolative moiety attached, although in some embodiments, more than one self-immolative moiety is attached as is described below (it should also be noted that other EAMs, as are broadly described herein, with self-immolative moieties can also be used). In some embodiments, when more than one self-immolative moiety is attached to a ferrocene, they are all attached to one of the cyclopentydienyl rings. In some embodiments, the self-immolative moieties are attached to different rings. In some embodiments, it is possible to saturate one or both of the cyclopentydienyl rings with self-immolative moieties, as long as one site is used for attachment to the electrode.

In some embodiments, the EAMs comprise substituted 1,1′-ferrocenes. Ferrocene is air-stable. It can be easily substituted with both TAM and anchoring group.

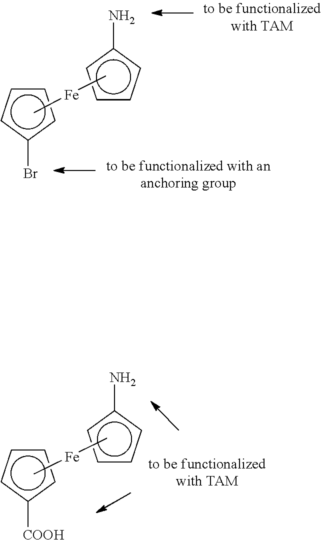

In some other embodiments, the EAMs comprise 1,3-disubstituted ferrocenes. 1,3-disubstituted ferrocenes are known (see, Bickert et al., Organometallics 1984, 3, 654-657; Farrington et al., Chem. Commun. 2002, 308-309; Pichon et al., Chem. Commun. 2004, 598-599; and Steurer et al., Organometallics 2007, 26, 3850-3859). In contrast to 1,1′-disubstituted ferrocenes where cyclopentadienyl (Cp) ring rotation can place both Cp substituents in an eclipsed conformation, 1,3-disubstituted ferrocene regioisomers provide a molecular architecture that enforces a rigid geometry between these Cp groups. Representative examples of 1,3-disubstituted ferrocenes are shown below such as compounds 1-5. An example of a 1,3-disubstituted ferrocene for attaching both anchoring and functional ligands is shown below:

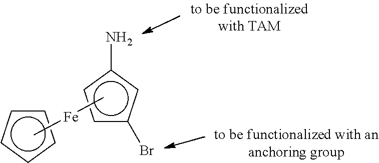

In addition in some embodiments, EAMs generally have an attachment moiety for attachment of the EAM to the conductive oligomer which is used to attach the EAM to the electrode. In general, although not required, in the case of metallocenes such as ferrocenes, the self-immolative moiety(ies) are attached to one of the cyclopentydienyl rings, and the attachment moiety is attached to the other ring, although attachment to the same ring can also be done. As will be appreciated by those in the art, any combination of self-immolative moieties and at least one attachment linker can be used, and on either ring.

In addition to the self-immolative moiety(ies) and the attachment moiety(ies), the ferrocene can comprise additional substituent groups, which can be added for a variety of reasons, including altering the E⁰ in the presence or absence of at least the self-immolative group. Suitable substituent groups, frequently depicted in associated and incorporated references as “R” groups, are recited in U.S. patent application Ser. No. 12/253,828, filed Oct. 17, 2008; U.S. patent application Ser. No. 12/253,875, filed Oct. 17, 2008; U.S. Provisional Patent Application No. 61/332,565, filed May 7, 2010; U.S. Provisional Patent Application No. 61/347,121, filed May 21, 2010; and U.S. Provisional Patent Application No. 61/366,013, filed Jul. 20, 2010, hereby incorporated by reference.

In some embodiments of any one of the methods or compositions provided herein, such as depicted below, the EAM does not comprise a self-immolative moiety, in the case where target analog moiety (TAM) is attached directly to the EAM and provides a change in E⁰ when the TAM is modified (removed/restructured) from the EAM by the enzyme.

In some embodiments of any one of the methods or compositions provided herein, the EAM can be introduced in solution for a homogeneous reaction competing with the target of interest and subsequently be detected after forming a self-assembled monolayer on an electrode.

In some embodiments of any one of the methods or compositions provided herein, the EAM can be attached to the electrode forming a self-assembled monolayer, followed by addition of the target of interest and the enzyme.

### Self-Immolative Moieties

In one embodiment of any one of the methods or compositions provided herein, the EAMs of the invention may include at least one self-immolative moiety that is covalently attached to the EAM such that the EAM has a first E⁰ when it is present and a second E⁰ when it has been removed as described below.

The term “self-immolative spacer” or “self-immolative linker” refers to a bifunctional chemical moiety that is capable of covalently linking two chemical moieties into a normally stable tripartate molecule. The self-immolative spacer is capable of spontaneously separating from the second moiety if the bond to the first moiety is cleaved. In the present invention, in some embodiments, the self-immolative spacer links a target analog moiety to the EAM. Upon exposure to an enzyme, the TAM is modified (removed/restructured) and the spacer falls apart. Generally speaking, any spacer where irreversible repetitive bond rearrangement reactions are initiated by an electron-donating alcohol functional group (i.e. quinone methide motifs) can be designed with boron groups serving as triggering moieties that generate alcohols under oxidative conditions. Alternatively, the boron moiety can mask a latent phenolic oxygen in a ligand that is a pro-chelator for a transition metal. Upon oxidation, the ligand can be transformed and initiate EAM formation in the SAM. For example, a sample chelating ligand is salicaldehyde isonicotinoyl hydrazone that binds iron.

As will be appreciated by those in the art, a wide variety of self-immolative moieties may be used with a wide variety of EAMs. Self-immolative linkers have been described in a number of references, including US Publication Nos. 20090041791; 20100145036 and U.S. Pat. Nos. 7,705,045 and 7,223,837, all of the description of which is expressly incorporated by reference in its entirety, particularly for the disclosure of self-immolative spacers.

### Electrodes

In some embodiments of any one of the methods or compositions provided herein the solid supports of the invention comprise electrodes. By “electrodes” herein is meant a composition, which, when connected to an electronic device, is able to sense a current or charge and convert it to a signal. Preferred electrodes are known in the art and include, but are not limited to, certain metals and their oxides, including gold, platinum, palladium, silicon, aluminum; metal oxide electrodes including platinum oxide, titanium oxide, tin oxide, indium tin oxide, palladium oxide, silicon oxide, aluminum oxide, molybdenum oxide (Mo2O6), tungsten oxide (WO3) and ruthenium oxides; and carbon (including glassy carbon electrodes, graphite, and carbon paste). Preferred electrodes include gold, silicon, carbon, and metal oxide electrodes, with gold being particularly preferred.

The electrodes described herein are generally depicted as a flat surface, which is only one of the possible conformations of the electrode and is for schematic purposes only. The conformation of the electrode will vary with the detection method used.

The electrodes of the invention can be incorporated into cartridges and can take a wide variety of configurations, and can include working and reference electrodes, interconnects (including “through board” interconnects), and microfluidic components. See for example U.S. Pat. No. 7,312,087, incorporated herein by reference in its entirety. In addition, in some embodiments, the chips generally include a working electrode with the components described herein, a reference electrode, and a counter/auxiliary electrode.

In a preferred embodiment, detection electrodes consist of an evaporated gold circuit on a polymer backing.

### Anchor Groups

The present invention in some embodiments provides compounds including the EAM (optionally become attached to the electrode surface with a conductive oligomer), the SAM, that become bound in situ to the electrode surface. Generally, in some embodiments, these moieties are attached to the electrode using anchor group. By “anchor” or “anchor group” herein is meant a chemical group that attaches the compounds of the invention to an electrode.

As will be appreciated by those in the art, the composition of the anchor group will vary depending on the composition of the surface to which it will be attached in situ. In the case of gold electrodes, both pyridinyl anchor groups and thiol based anchor groups find particular use.

The covalent attachment of the conductive oligomer may be accomplished in a variety of ways, depending on the electrode and the conductive oligomer used. Generally, some type of linker is used, as depicted below as “A” in Structure 1, where X is the conductive oligomer, and the hatched surface is the electrode:

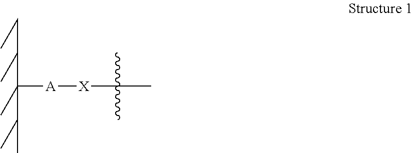

In this embodiment, A is a linker or atom. The choice of “A” will depend in part on the characteristics of the electrode. Thus, for example, A may be a sulfur moiety when a gold electrode is used. Alternatively, when metal oxide electrodes are used, A may be a silicon (silane) moiety attached to the oxygen of the oxide (see for example Chen et al., Langmuir 10:3332-3337 (1994); Lenhard et al., J. Electroanal. Chem. 78:195-201 (1977), both of which are expressly incorporated by reference). When carbon based electrodes are used, A may be an amino moiety (preferably a primary amine; see for example Deinhammer et al., Langmuir 10:1306-1313 (1994)). Thus, preferred A moieties include, but are not limited to, silane moieties, sulfur moieties (including alkyl sulfur moieties), and amino moieties.

In some embodiments, the electrode is a carbon electrode, i.e. a glassy carbon electrode, and attachment is via a nitrogen of an amine group. A representative structure is depicted in Structure 15 of US Patent Application Publication No. 20080248592, hereby incorporated by reference in its entirety but particularly for Structures as described therein and the description of different anchor groups and the accompanying text. Again, additional atoms may be present, i.e. linkers and/or terminal groups.

In Structure 16 of US Patent Application Publication No.20080248592, hereby incorporated by reference as above, the oxygen atom is from the oxide of the metal oxide electrode. The Si atom may also contain other atoms, i.e., be a silicon moiety containing substitution groups. Other attachments for SAMs to other electrodes are known in the art; see for example Napier et al., Langmuir, 1997, for attachment to indium tin oxide electrodes, and also the chemisorption of phosphates to an indium tin oxide electrode (talk by H. Holden Thorpe, CHI conference, May 4-5, 1998).

In one preferred embodiment, indium-tin-oxide (ITO) is used as the electrode, and the anchor groups are phosphonate-containing species.

### Sulfur Anchor Groups

Although depicted in Structure 1 as a single moiety, the conductive oligomer may be attached to the electrode with more than one “A” moiety; the “A” moieties may be the same or different. Thus, for example, when the electrode is a gold electrode, and “A” is a sulfur atom or moiety, multiple sulfur atoms may be used to attach the conductive oligomer to the electrode, such as is generally depicted below in Structures 2, 3 and 4. As will be appreciated by those in the art, other such structures can be made. In Structures 2, 3 and 4 the A moiety is just a sulfur atom, but substituted sulfur moieties may also be used.

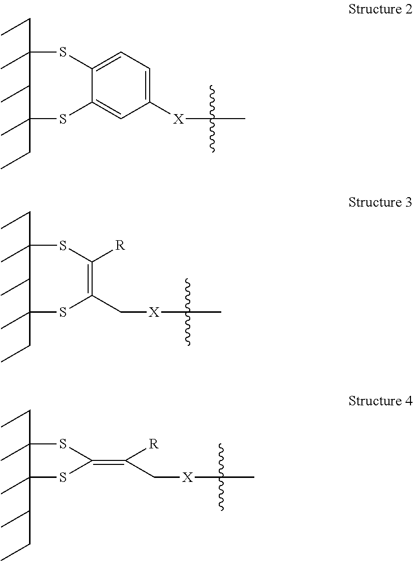

It should also be noted that similar to Structure 4, it may be possible to have a conductive oligomer terminating in a single carbon atom with three sulfur moieties attached to the electrode.

In another aspect, the present invention provides anchors comprising conjugated thiols. In some embodiments, the anchor comprises an alkylthiol group.

In another aspect, the present invention provides conjugated multipodal thio-containing compounds that serve as anchoring groups in the construction of electroactive moieties for analyte detection on electrodes, such as gold electrodes. That is, spacer groups (which can be attached to EAMs or an “empty” monolayer forming species) are attached using two or more sulfur atoms. These multipodal anchor groups can be linear or cyclic, as described herein.

In some embodiments, the anchor groups are “bipodal”, containing two sulfur atoms that will attach to the gold surface, and linear, although in some cases it can be possible to include systems with other multipodalities (e.g. “tripodal”). Such a multipodal anchoring group can display increased stability and/or allow a greater footprint for preparing SAMs from thiol-containing anchors with sterically demanding headgroups.

In some embodiments, the anchor comprises cyclic disulfides (“bipod”). Although in some cases it can be possible to include ring system anchor groups with other multipodalities (e.g. “tripodal”). The number of the atoms of the ring can vary, for example from 5 to 10, and also includes multicyclic anchor groups, as discussed below

In some embodiments, the anchor groups comprise a [1,2,5]-dithiazepane unit which is seven-membered ring with an apex nitrogen atom and a intramolecular disulfide bond as shown below:

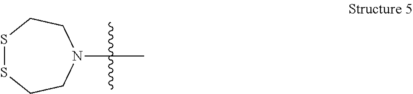 [SMILES: *N1CCSSCC1]

In Structure (5), it should also be noted that the carbon atoms of the ring can additionally be substituted. As will be appreciated by those in the art, other membered rings can also be included. In addition, multicyclic ring structures can be used, which can include cyclic heteroalkanes such as the [1,2,5]-dithiazepane shown above substituted with other cyclic alkanes (including cyclic heteroalkanes) or aromatic ring structures.

In some embodiments, the anchor group and part of the spacer has the structure shown below

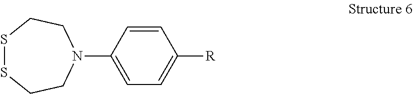

The “R” group herein can be any substitution group, including a conjugated oligophenylethynylene unit with terminal coordinating ligand for the transition metal component of the EAM.

The anchors can be synthesized from a bipodal intermediate (I) (the compound as formula III where R═I), which is described in Li et al., Org. Lett. 4:3631-3634 (2002), herein incorporated by reference. See also Wei et al, J. Org, Chem. 69:1461-1469 (2004), herein incorporated by reference.

The number of sulfur atoms can vary as outlined herein, with particular embodiments utilizing one, two, and three per spacer.

As will be appreciated by those in the art, the compositions of the invention can be made in a variety of ways. In some embodiments, the composition are made according to methods disclosed in U.S. Pat. Nos. 6,013,459, 6,248,229, 7,018,523, 7,267,939, etc., all of which are herein incorporated in their entireties for all purposes.

### Applications

The systems of the invention find use in the detection of a variety of target analytes, as outlined herein. In particular, the systems of the invention find great use in the detection of molecules for which traditional capture ligands may not be available or enzymatic products are not readily used in further reactions to product a detectible signal.

Additionally, it is possible to detect multiple targets simultaneously without requiring any segregation of the sample. Each electrode, in an array of electrodes, of any one of the methods or compositions provided herein can be modified with a specifically designed EAM, comprising a target specific TAM attached to the transition metal complex of the EAM which could in turn react with an enzyme, for which both the target and the target analog (TAM) are substrates, when two or more enzymes, which are each selective for respective target/target analog pairs, are introduced. The enzyme will react in a concentration dependent manner, e.g., if more target is present, more target will be reacted and less electro-active target analog will be reacted, if less target is present then the opposite will be true.

The TAM can be modified (removed/restructured) by the enzyme specific for the respective target/target analog pair to provide the specific EAM with a unique redox potential specific to one target. With multiple EAMs, each modified with a unique target specific TAM, each may generate an electrochemical signal at a distinct potential, each signal corresponding to specific reacted EAMs, and a specific target. Therefore targets) could be detected simultaneously.

In one embodiment of any one of the methods or compositions provided herein, the above detection can be carried out in a solution phase assay mixture, contacting the target with the EAM and the enzyme in the solution phase, where the target and the enzyme can be contacted with EAM simultaneously or sequentially, where target is contacted to the EAM first followed by enzyme addition. Later, the assay mixture containing reacted and unreacted EAM can be delivered to an electrode for SAM formation and detection.

In another embodiment of any one of the methods or compositions provided herein, the target is contacted with the EAM (comprising TAM) which is covalently attached to the electrode, followed by the addition of the enzyme (for which the target and TAM are substrates) either simultaneously or after the target has been introduced.

In some embodiments of any one of the methods or compositions provided herein, assay conditions mimic physiological conditions. In some embodiments of any one of the methods provided herein a plurality of assay mixtures are run in parallel with different concentrations to obtain a differential response to the various concentrations. That is, a dose response curve can be generated in any one of the methods provided herein. In some embodiments of any one of these methods, one of the concentrations serves as a negative control, i.e., at zero concentration or below the level of detection. Once a dose response has been established with known quantities, it can be used to measure unknown quantities in samples. In addition, as will be appreciated by those in the art, any variety of other reagents may be included in the assays. These include reagents like salts, buffers, detergents, neutral proteins, e.g. albumin, etc. which may be used to facilitate optimal reactions or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

### Detection

Electron transfer between the redox active molecule and the electrode can be detected in a variety of ways, with electronic detection, including, but not limited to, amperommetry, voltammetry, capacitance and impedance being preferred. These methods include time or frequency dependent methods based on AC or DC currents, pulsed methods, lock in techniques, and filtering (high pass, low pass, band pass). In some embodiments of any one of the methods provided, all that is required is electron transfer detection; in others, the rate of electron transfer may be determined.

In some embodiments, electronic detection is used, including amperommetry, voltammetry, capacitance, and impedance. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry, and photoelectrochemistry.

In some embodiments, monitoring electron transfer is via amperometric detection. This method of detection involves applying a potential (as compared to a separate reference electrode) between the electrode containing the compositions of the invention and an auxiliary (counter) electrode in the test sample. Electron transfer of differing efficiencies can be induced in samples in the presence or absence of target analyte.

The device for measuring electron transfer amperometrically can involve sensitive current detection and includes a means of controlling the voltage potential, usually a potentiostat. This voltage can be optimized with reference to the potential of the redox active molecule.

In some embodiments, alternative electron detection modes are utilized. For example, potentiometric (or voltammetric) measurements involve non faradaic (no net current flow) processes and are utilized traditionally in pH and other ion detectors. Similar sensors can be used to monitor electron transfer between the redox active molecules and the electrode. In addition, other properties of insulators (such as resistance) and of conductors (such as conductivity, impedance and capacitance) could be used to monitor electron transfer between the redox active molecules and the electrode. Finally, any system that generates a current (such as electron transfer) can also generate a small magnetic field, which may be monitored in some embodiments.

It should be understood that one benefit of the fast rates of electron transfer observed in some embodiments of the compositions and methods of the invention is that time resolution can greatly enhance the signal to noise results of monitors based on electronic current. The fast rates of electron transfer of the present invention can result both in high signals and stereotyped delays between electron transfer initiation and completion. By amplifying signals of particular delays, such as through the use of pulsed initiation of electron transfer and “lock in” amplifiers of detection, orders of magnitude improvements in signal to noise may be achieved.

In some embodiments, electron transfer is initiated and detected using direct current (DC) techniques. As noted above, the first E⁰ of the redox active molecule before and the second E⁰ of the reacted redox active molecule afterwards can allow the detection of the analyte. As will be appreciated by those in the art, a number of suitable methods may be used to detect the electron transfer.

In some embodiments, electron transfer is initiated using alternating current (AC) methods. A first input electrical signal is applied to the system, preferably via at least the sample electrode (containing the complexes of the invention) and the counter electrode, to initiate electron transfer between the electrode and the second electron transfer moiety. Three electrode systems may also be used, with the voltage applied to the reference and working electrodes. In this embodiment, the first input signal comprises at least an AC component. The AC component may be of variable amplitude and frequency. Generally, for use in the present methods, the AC amplitude ranges from about 1 mV to about 1.1 V, with from about 10 mV to about 800 mV being preferred, and from about 10 mV to about 500 mV being especially preferred. The AC frequency ranges from about 0.01 Hz to about 10 MHz, with from about 1 Hz to about 1 MHz being preferred, and from about 1 Hz to about 100 kHz being especially preferred.

In some embodiments, the first input signal comprises a DC component and an AC component. That is, a DC offset voltage between the sample and counter electrodes is swept through the electrochemical potential of the second electron transfer moiety. The sweep is used to identify the DC voltage at which the maximum response of the system is seen. This is generally at or about the electrochemical potential of the redox active molecule. Once this voltage is determined, either a sweep or one or more uniform DC offset voltages may be used. DC offset voltages of from about 1 V to about +1.1 V are preferred, with from about 500 mV to about +800 mV being especially preferred, and from about 300 mV to about 500 mV being particularly preferred. On top of the DC offset voltage, an AC signal component of variable amplitude and frequency can be applied. If the redox active molecule has a low enough solvent reorganization energy to respond to the AC perturbation, an AC current will be produced due to electron transfer between the electrode and the redox active molecule.

In some embodiments, the AC amplitude is varied. Without being bound by theory, it appears that increasing the amplitude increases the driving force. Thus, higher amplitudes, which result in higher overpotentials can give faster rates of electron transfer. Thus, generally, the same system gives an improved response (i.e. higher output signals) at any single frequency through the use of higher overpotentials at that frequency. Thus, the amplitude may be increased at high frequencies to increase the rate of electron transfer through the system, resulting in greater sensitivity. In addition, as noted above, it may be possible to the first and second E⁰ of the redox active molecules, molecules on the basis of the rate of electron transfer, which in turn can be used either to distinguish the two on the basis of frequency or overpotential.

In some embodiments, measurements of the system are taken at least two separate amplitudes or overpotentials, with measurements at a plurality of amplitudes being preferred. As noted above, changes in response as a result of changes in amplitude may form the basis of identification, calibration and quantification of the system.

In some embodiments, the AC frequency is varied. At different frequencies, different molecules can respond in different ways. As will be appreciated by those in the art, increasing the frequency generally increases the output current. However, when the frequency is greater than the rate at which electrons may travel between the electrode and the redox active molecules, higher frequencies result in a loss or decrease of output signal. At some point, the frequency will be greater than the rate of electron transfer through even solvent inhibited redox active molecules, and then the output signal will also drop.

In addition, the use of AC techniques can allow for the significant reduction of background signals at any single frequency due to entities other than the covalently attached nucleic acids, i.e., “locking out” or “filtering” unwanted signals. That is, the frequency response of a charge carrier or redox active molecule in solution can be limited by its diffusion coefficient. Accordingly, at high frequencies, a charge carrier may not diffuse rapidly enough to transfer its charge to the electrode, and/or the charge transfer kinetics may not be fast enough. This is particularly significant in embodiments that do not utilize a passivation layer monolayer or have partial or insufficient monolayers, i.e., where the solvent is accessible to the electrode. However, using the present AC techniques, one or more frequencies can be chosen that prevent a frequency response of one or more charge carriers in solution, whether or not a monolayer is present. This is particularly significant since many biological fluids such as blood contain significant amounts of redox active molecules which can interfere with amperometric detection methods.

In some embodiments, measurements of the system are taken at least two separate frequencies, with measurements at a plurality of frequencies being preferred. A plurality of frequencies includes a scan. In a preferred embodiment, the frequency response is determined at least two, preferably at least about five, and more preferably at least about ten frequencies.

### Signal Processing

After transmitting the input signal to initiate electron transfer, an output signal can be received or detected. The presence and magnitude of the output signal can depend on the overpotential/amplitude of the input signal; the frequency of the input AC signal; the composition of the intervening medium, i.e. the impedance, between the electron transfer moieties; the DC offset; the environment of the system; and the solvent. At a given input signal, the presence and magnitude of the output signal can depend in general on the solvent reorganization energy required to bring about a change in the oxidation state of the metal ion. Thus, upon transmitting the input signal, comprising an AC component and a DC offset, electrons can be transferred between the electrode and the redox active molecule, when the solvent reorganization energy is low enough, the frequency is in range, and the amplitude is sufficient, resulting in an output signal.

In some embodiments, the output signal comprises an AC current. As outlined above, the magnitude of the output current can depend on a number of parameters. By varying these parameters, the system may be optimized in a number of ways.

In general, AC currents generated in the present invention can range from about 1 femptoamp to about 1 milliamp, with currents from about 50 femptoamps to about 100 microamps being preferred, and from about 1 picoamp to about 1 microamp being especially preferred.

### Apparatus

The present invention further provides apparatus for the detection of analytes using the methods provided herein, including AC detection methods. The apparatus can include a test chamber which has at least a first measuring or sample electrode, and a second measuring or counter electrode. Three electrode systems are also useful. The first and second measuring electrodes can be in contact with a test sample receiving region, such that in the presence of a liquid test sample, the two electrodes may be in electrical contact.

In yet another embodiment, the first measuring electrode comprises a redox active complex, covalently attached via a spacer, and preferably via a conductive oligomer, such as are described herein. Alternatively, the first measuring electrode can comprise covalently attached redox active molecules and TAM.

The apparatus can further comprise a voltage source electrically connected to the test chamber; that is, to the measuring electrodes. Preferably, the voltage source is capable of delivering AC and DC voltages, if needed.

In an embodiment, the apparatus further comprises a processor capable of comparing the input signal and the output signal. The processor is coupled to the electrodes and configured to receive an output signal, and thus detect the presence of the target analyte.

### EXAMPLES

### Example 1

### Tyrosine-EAM Dose Response

Purpose: To detect and measure the target analyte Tyr-ethyl-ester using chymotrypsin and an EAM comprising a tyrosine TAM in a solution-based competitive enzymatic assay format; to create a dose response with points in triplicate.

### Method

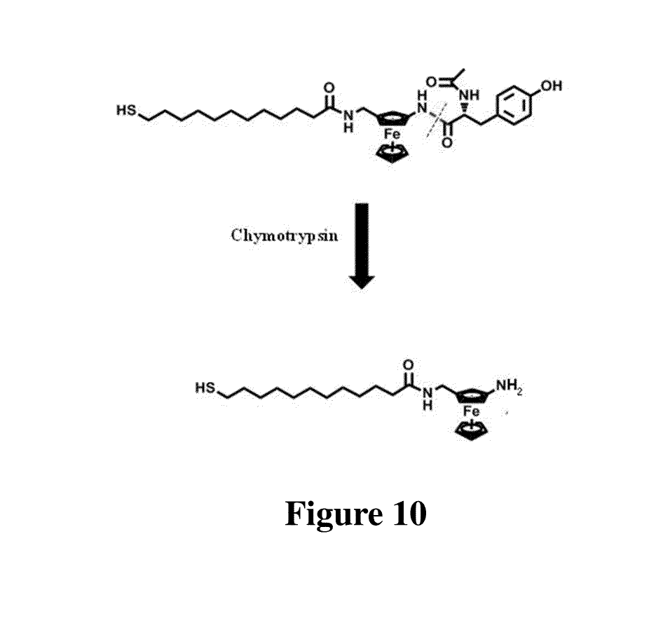

### Reagent Prep

| Materials | Concentration | Amount | Incubation time |
|---|---|---|---|
| Chymotrypsin | 5 uM, (final) | 90 uL | 10 min reaction |
| in PBS pH 7.4 | | | |
| Tyr-ethyl-ester | 30 uM, 90 uM, | 5 uL | — |
| in EtOH | 270 uM, 810 uM, | | |
| | 2.4 mM, 7.3 mM, | | |
| | 22 mM (final) | | |
| EAM RN5-87B | 50 uM EAM, | 5 uL | 5 min SAM |
| (FIG. 10) | 100 uM diluent | | formation |

### Experimental Procedure

5 uL EAM was added to each tube.

5 uL Tyr-ethyl-ester target (varying concentrations) was added to the EAM.

90 uL 5 uM Chymotrypsin was added to each tube for a reaction time of 10 minutes. Solution was mixed.

Entire solution was added to gold electrode chip and incubated for 5 min to allow SAM formation.

Electrode chips were washed 4× with 1M LiClO₄

Each electrode chip was scanned using CHI potentiostat.

### Results

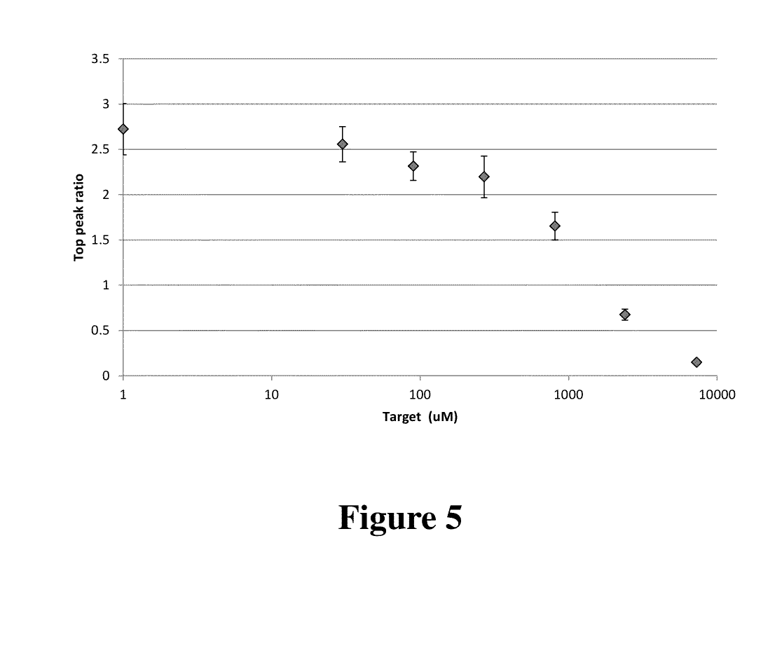

The results of this example are summarized in the graph in FIG. 5. A dose response was successfully obtained for the target Tyrosine-ethyl-ester, a substrate for chymotrypsin. Each target concentration was run in triplicate and FIG. 5 includes the standard deviation error bars. As it is a competitive assay in which the added chymotrypsin also reacts with the EAM (which has a tyrosine TAM attached to the end), the dose response for the target has a negative relationship.

### Example 2

### Tyrosine-EAM Dose Response for Multiple Targets

Purpose: To detect and measure two different targets, Lys-Tyr-Lys acetate salt and N-Benzoyl-L-tyrosine ethyl ester, and obtain a dose response for each. Test was done according to a solution-based competitive enzymatic assay format using chymotrypsin and an EAM comprising a tyrosine TAM. For Target 2, the test was run at two different enzyme concentrations.

### Method

### Reagent Prep

| Materials | Concentration | Amount | | Incubation time |
|---|---|---|---|---|
| Chymotrypsin | 1.25-5 uM [Final] | 45-90 | uL | 20 mins with |
| (enzyme) | | | | Target |
| Target 1: | 1.6 mM, 800 uM, 400 uM, | 45 | uL | 20 mins with |
| Lys-Tyr-Lys | 200 uM, 100 uM, 50 uM | | | enzyme |
| acetate salt | [Final concentrations] | | | |
| in PBS | (MW = 437.53) | | | |
| Target 2: | 8 mM, 4 mM, 2 mM, | 5 | uL | 20 mins with |
| N-Benzoyl-L- | 1 mM, 500 uM, 250 uM, | | | enzyme |
| tyrosine ethyl | 125 uM [Final | | | |
| ester in EtOH | concentrations] | | | |
| | (MW = 313.35) | | | |
| EAM solution: | 500 uM EAM (MW = | 5-10 | uL | 5 minute SAM |
| EAM RN5_87 | 635.64) | | | formation time |
| (FIG. 10) | 1 mM C₁₆ Diluent; 1 mM | | | |
| | EAM | | | |
| | 2 mM C₁₆ Diluent | | | |

### Experimental Procedure

### Target 1: Lys-Tyr-Lys Titration:

45 uL Target 1 (varying concentrations in PBS) was added to microcentrifuge tube containing 10 uL of EAM solution (500 uM EAM 1 mM Diluent).

45 uL 11.11 uM Chymotrypsin was added to each tube containing target and EAM solution for a 20 minute reaction (final enzyme concentration of 5 uM).

Reaction solution was added to electrode chip for a 5 min SAM formation.

Electrode washed 4× with 1M LiClO₄ and chips tested with potentiostat.

Target 2 First Enzyme Concentration: N-Benzoyl-L-Tyr-Ethyl-Ester Titration with 5 uM Chymo:

5 uL Target 2 (varying concentrations in EtOH) was added to microcentrifuge tube containing 5 uL EAM solution (1 mM EAM 2 mM Diluent).

90 uL 5.56 uM Chymotrypsin was added to each tube containing target and EAM solution for a 20 minute reaction (final enzyme concentration of 5 uM).

Reaction solution was added to electrode chip for a 5 min SAM formation.

Electrode was washed 4× with 1M LiClO₄ and chips were tested with potentiostat.

Target 2 Second Enzyme Concentration: N-Benzoyl-L-Tyr-Ester Titration with 1.25 uM Chymo:

5 uL Target 2 (varying concentrations in EtOH) was added to microcentrifuge tube containing 5 uL EAM solution (1 mM EAM 2 mM Diluent).

90 uL 1.39 uM Chymotrypsin was added to each tube containing target and EAM solution for a 20 minute reaction (final enzyme concentration of 1.25 uM).

Reaction solution was added to electrode chip for a 5 min SAM formation.

Electrode was washed 4× with 1M LiClO₄ and chips were tested with potentiostat.

### Results

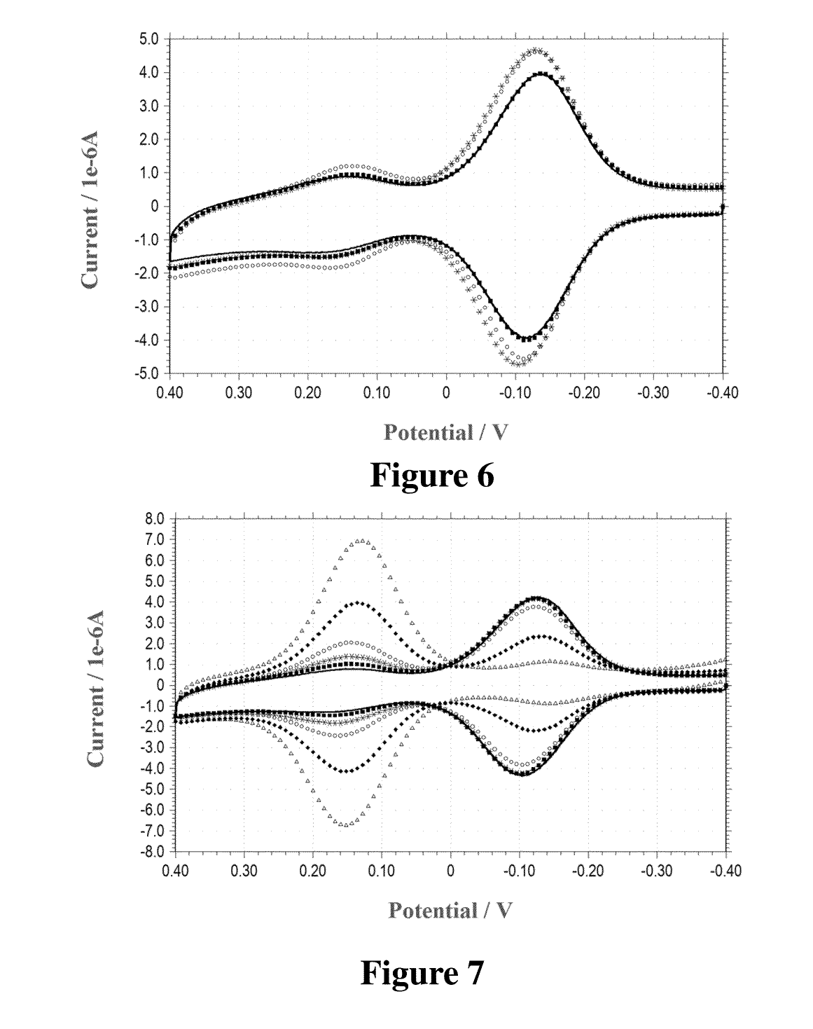

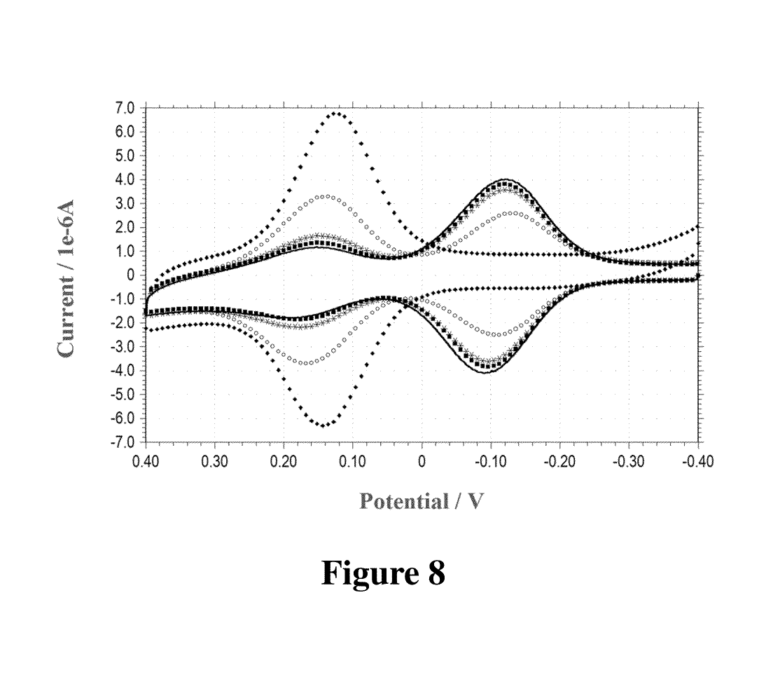

The results of this example are summarized in FIGS. 7, 8, and 9, each of which depicts a plot of voltage vs. current obtained during measurement of the electrode chip with a potentiostat. Results for experimental procedure 2i are shown in FIG. 6. FIG. 6 shows the response of various concentrations of Target 1, the Lys-Tyr-Lys substrate, with 5 uM Chymotrypsin in a competitive enzymatic assay with the EAM comprising a tyrosine TAM (see FIG. 10 for structure). In FIG. 6, Line: 0 uM Lys-Tyr-Lys, Square: 50 uM Lys-Tyr-Lys, Asterisk: 200 uM Lys-Tyr-Lys, Circle: 800 uM Lys-Tyr-Lys. The results show that a differential signal is seen higher target concentrations than lower target concentrations, though the separation may be improved with further optimization of assay conditions. Results for experimental procedure 2ii are shown in FIG. 7. FIG. 7 shows the response of various concentrations of Target 2, Tyrosine ethyl ester substrate, with 5 uM Chymotrypsin in a competitive enzymatic assay with the EAM comprising a tyrosine TAM (see FIG. 10 for structure). A differential signal is produced for each concentration tested, though separation may be improved with further optimization of assay conditions. Results for experimental procedure 2iii are shown in FIG. 8. FIG. 8 shows the response of various concentrations of Target 2, Tyrosine ethyl ester substrate, with 1.25 uM Chymotrypsin in a competitive enzymatic assay with the EAM comprising a tyrosine TAM (see FIG. 10 for structure). After decreasing the enzyme concentration 4× to 1.25 uM, there is much better separation of the peaks, and each target concentration produces a clearly distinct signal.

### Example 3

### Tyrosine-EAM Dose Response Repeated

Purpose: To test chymotrypsin concentrations and incubation times on the tyrosine ethyl ester substrate to determine the best parameters for a lower LOD using target/enzyme/EAM reagents that already have been given in other examples.

### Method

### Reagent Prep

| Materials | Concentration | Amount | Incubation time |
|---|---|---|---|
| Chymotrypsin | 312.5 nM, 625 nM, 5 uM | 90 uL | 20 mins |
| in PBS | [Final] | | |
| Target: | 8 mM, 4 mM, 2 mM, | 5 uL | |
| N-Benzoyl-L- | 1 mM, 500 uM, 250 uM, | | |
| tyrosine ethyl | 125 uM, 62.5 uM, | | |
| ester in EtOH | 31.25 uM [Final] | | |
| | (MW = 313.35) | | |
| EAM solution: | 50 uM EAM (MW = | 5 uL | 5 minute SAM |
| EAM RN5_87 | 635.64) | | formation time |
| (FIG. 10) | 100 uM C16 DIluent | | |

### Experimental Procedure

5 uL of target (varying concentrations in EtOH) was added to microcentrifuge tube containing 5 uL EAM Solution.

90 uL 347.2 nM Chymotripsin was added to each tube containing target and EAM solution for a 20 min reaction (final enzyme concentration 12.5 nM).

Reaction solution was added to electrode chip for 5 min SAM formation.

Electrode was washed 4× with 1M LiClO₄ and electrode chips tested.

### Results

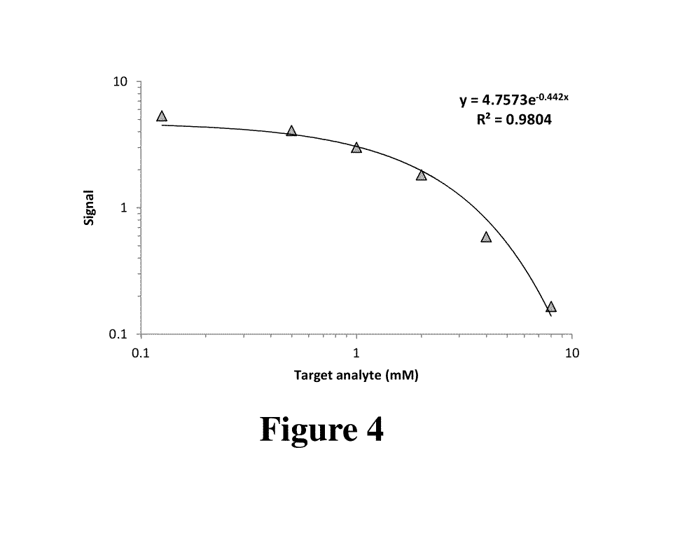

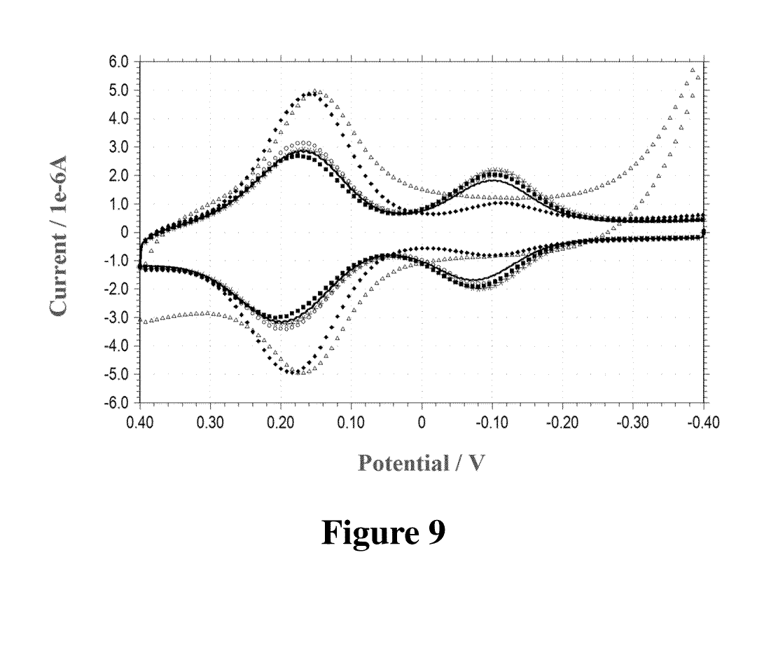

The results of this example are summarized in FIG. 9 and FIG. 4. FIG. 10 shows a graph of the potential vs current when testing the electrode chips in this experiment. Differential signal can be seen for target concentrations. The lowest concentration of tyrosine that could be detected was 2 mM (the 500 uM was approximately equal to the 0 uM). Line: 0 uM Tyrosine, Square: 31.25 uM Tyrosine, Asterisk: 125 uM Tyrosine, Circle: 500 uM Tyrosine, Diamond: 2 mM Tyrosine, Triangle: 8 mM Tyrosine with 312.5 nM Chymotrypsin, 20 min reaction/5 min SAM formation time. FIG. 4 shows the same data output transformed to clearly show the dose response obtained. The graph shows a clear inverse relationship between target concentration and signal generated, as well as good fit for the dose response curve.

### Example 4

### Synthesis of Tyrosine-EAM (EAM Molecule Comprising an Anchor Group, Readox Active Complex, and Target Analog Moiety TAM

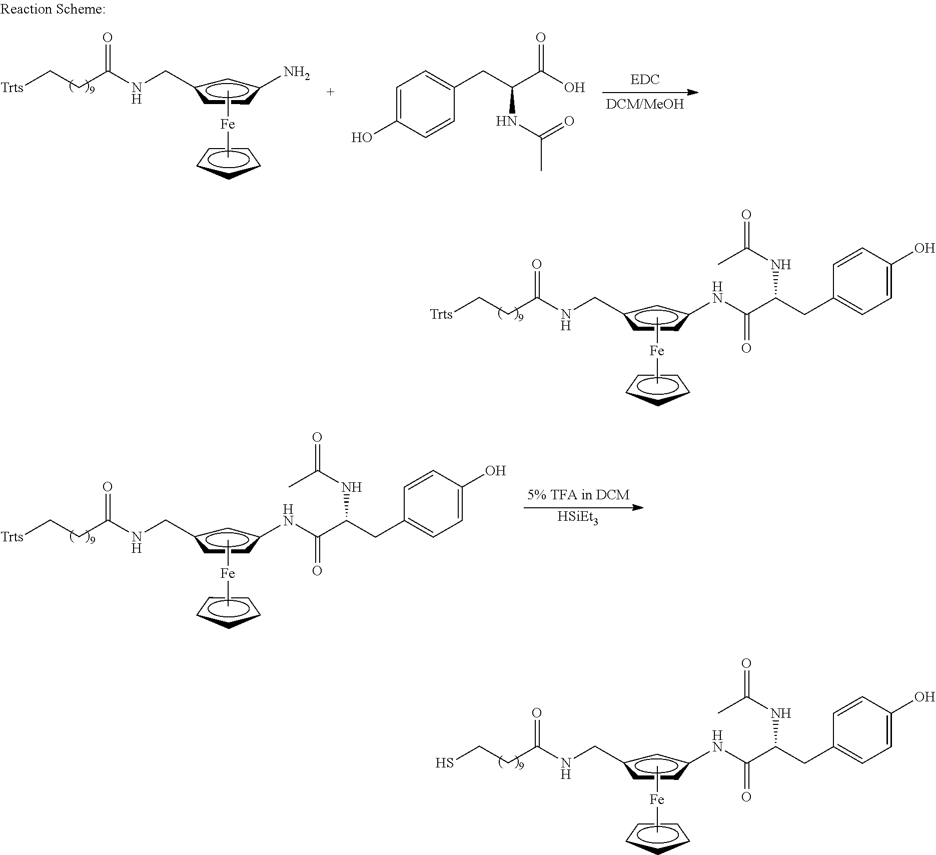

Coupling 1,3-Ferrocene Amine with N-Acetyl-Tyrosine:

A trityl protected 1,3-ferrocene amine can be synthesized as described in the art (see, for example, US20130112572A1 Example 2 structure 3).

Into a 25 ml Schlenk flask was added trityl protected 1,3-ferrocene amine and dichloromethane (DCM) (1 ml). The solution was degassed for 5 min. A solution of 2 molar equivalent N-acetyl-tyrosine in methanol (1 ml) was degassed and added to the Schlenk flask. 2 molar equivalent EDC was added and the reaction was set to stir. The reaction progress was checked by thin layer chromatography after 2.5 hours (0.3:3.7:6 methanol:Et₂O:DCM) and found to be complete. Contents were concentrated under vacuum to a brown/yellow oil.

Crude product was purified by column chromatography using gradient solvent system (5%-50% of MeOH in DCM) as an eluent to collect three fractions including the baseline. The fraction with R_{f} value approximately 0.3 was analyzed by mass spectrometry (ESI, calculated: 877.36, found: 877.51) and NMR (18 mg, 36%). ¹H and ¹³C NMR also matched the title compound (ferrocene amine coupled with N-acetyl-tyrosine). Hence, a tyrosine-EAM was successfully prepared.

Trityl Deprotect EAM-Tyrosine:

Into a 25 ml Schlenk flask was added ferrocene amine coupled with N-acetyl-tyrosine from previous step and DCM (1 ml). The solution was degassed. In a conical vial, was added triethylsilene (5 molar equivalent), trifluoroacetic acid (100 uL of 5% in DCM), and DCM (1 ml). The solution was degassed and transferred to the Schlenk flask via cannula. The reaction was set to stir under argon. After 2 hours, the reaction progress was checked by thin layer chromatography using 50% methanol:DCM and found to be complete. The reaction contents were concentrated under vacuum to a yellow/brown residue. This was further purified by column chromatography using 5% methanol:DCM as an eluent to yield a yellow oil (13.4 mg, and 99% purity). The ¹H and ¹³C NMR confirmed production of tyrosine-EAM.

We claim:

1. A method for detecting a target analyte in a test sample, said method comprising:
(a) contacting the test sample with an electroactive moiety (EAM) and at least one enzyme or contacting the test sample with a solid support, said solid support comprising an electrode or an array of electrodes, said electrode comprising:
(i) a self-assembled monolayer; and
(ii) a covalently attached electroactive moiety (EAM), wherein:
the EAM comprises a transition metal complex and an target analog moiety (TAM), the target analyte and the target analog moiety are substrates of the at least one enzyme, and the EAM has a first E^{o} when the TAM has not been modified by the at least one enzyme and a second E^{o} when at least a portion of the TAM has been modified by the at least one enzyme;
(b) detecting a change between the first E^{o} and the second E^{o} of the EAM, wherein the change is an indication of the presence of said at least one target analyte; and
(c) determining the concentration of the target analyte.

2. A method according to claim 1, wherein an assay mixture in a solution phase is formed in step (a) and prior to step (b).

3. A method according to claim 1, further comprising:
contacting said assay mixture with a solid support comprising an electrode or an array of electrodes, under conditions such that a self-assembled monolayer (SAM) forms on said electrode.

4. A method for detecting a target analyte in a test sample, said method comprising:
(a) contacting the test sample with an electroactive moiety (EAM) and at least one enzyme to form an assay mixture in solution phase, wherein:
the EAM comprises a transition metal complex and an target analog moiety (TAM), the target analyte and the target analog moiety are substrates of the at least one enzyme, and
the EAM has a first E^{o} when the TAM has not been modified by the at least one enzyme and a second E^{o} when at least a portion of the TAM has been modified by the enzyme;
(b) contacting said assay mixture with a solid support comprising an electrode or an array of electrodes under conditions such that a self-assembled monolayer (SAM) forms on said electrode;
(c) detecting for a change between the first E^{o} and the second E^{o} of said EAM, wherein said change is an indication of the presence of said target analyte; and
(d) determining the concentration of the target analyte.

5. A method according to claim 4, wherein said EAM is covalently attached to the electrode or the array of electrodes on the solid support as the self-assembled monolayer (SAM).

6. (canceled)

7. A method according to claim 1, wherein said EAM further comprising a self-immolative moiety (SIM) which joins said TAM to said transition metal complex.

8. A method according to claim 1, wherein said at least one enzyme is selected from the group consisting of proteases, peptidases, phosphatases, oxidases, hydrolases, lyases, transferases, isomerase, ligases, and ligases.

9. A method according to claim 1, wherein said transition metal complex comprises a transition metal selected from the group consisting of iron, ruthenium, and osmium.

10. A method according to claim 1, wherein said transition metal complex comprises a ferrocene and substituted ferrocene.

11. A method according to claim 4, wherein said EAM comprises a flexible oligomer anchor tethering said transition metal complex to said electrode.

12. The method of claim 11, wherein said flexible anchor comprises a hydrophobic oligomer comprising side chains that limit intermolecular hydrophobic interactions and prevent organization and rigidity.

13. A method according to claim 4, wherein said EAM comprises a flexible oligomer anchor tethering said transition metal complex to said electrode.

14. The method of claim 13, wherein said flexible anchor comprises an oligomer comprising polar and/or charged functional groups.

15. The method of claim 13, wherein said flexible oligomer anchor tethering said transition metal complex to said electrode comprises poly acrylic acid, polyethylene glycol (PEG), poly vinyl alcohol, polymethacrylate, poly vinylpyrrolidinone, acrylamide, maleic anhydride, poly vinylpyridine, allylamine, ethyleneimine, or oxazoline.

16. A method according to claim 4, wherein the electrodes in said array of electrodes are modified with a SAM and wherein at least some of the electrodes comprise a different EAM and TAM from another electrode.

17. A method according to claim 16, wherein the different TAMs are substrates for different enzymes.

18. A method according to claim 16, further comprising detecting two or more different target analytes in said test sample using two or more enzymes.

19. A composition comprising:
a solid support comprising an electrode comprising:
(i) a self-assembled monolayer (SAM); and
(ii) a covalently attached electroactive active moiety (EAM) comprising a transition metal complex and an target analog moiety (TAM), wherein said EAM has a first E⁰ when said TAM is present and a second E⁰ when said TAM is modified.

20. A composition according to claim 19, wherein the EAM comprises a self-immolative moiety (SIM) that joins the TAM to the transition metal complex.

21. A composition according to claim 19, wherein the transition metal complex comprises a transition metal selected from the group consisting of iron, ruthenium, and osmium.

22-29. (canceled)
